(19) United States
(12) Patent Application Publication
(10) Pub. No.: US 2008/0131395 A1
(43) Pub. Date: Jun. 5, 2008

(54) CONTROLLED GAS RELEASE FROM A MELT PROCESSABLE COMPATIBLE POLYMER BLEND

(75) Inventors: Stephen T. Wellinghoff, San Antonio, TX (US); Joel J. Kampa, Lakehills, TX (US); Douglas P. Hanson, San Antonio, TX (US); Michael John Finnegan, Columbia, SC (US)

Correspondence Address: SENNIGER POWERS LLP, ONE METROPOLITAN SQUARE, 16TH FLOOR, ST LOUIS, MO, 63102, US

(73) Assignees: MICROACTIVE CORP., Reno, NV (US); SOUTHWEST RESEARCH INSTITUTE, San Antonio, TX (US)

(21) Appl. No.: 11/945,583

(22) Filed: Nov. 27, 2007

Related U.S. Application Data
(60) Provisional application No. 60/867,303, filed on Nov. 27, 2006.

(51) Int. Cl. A61L 9/01; C08L 55/00; A01N 31/00; A01P 3/00; A01P 1/00

(52) U.S. Cl. 424/76.3; 525/55; 525/540; 424/78.08

(57) ABSTRACT

The invention relates generally to compatible polymer blends that can be extruded or injection molded into films or other objects that will generate and release a gas such as sulfur dioxide, carbon dioxide, or chlorine dioxide upon contact with moisture.

### REFERENCE TO RELATED APPLICATIONS

This application claims the benefit of U.S. Provisional Application No. 60/867,303 filed Nov. 27, 2006.

### FIELD OF THE INVENTION

The present invention relates generally to polymeric alloy compositions that can be extruded or injection molded into films or other objects that will release a gas such as sulfur dioxide, carbon dioxide, or chlorine dioxide upon contact with moisture. The invention particularly relates to polymer blends containing chlorite anions capable of reacting with hydronium ions to generate chlorine dioxide gas. Films or objects containing such ions may be used for retarding, controlling, killing or preventing microbiological contamination from bacteria, fungi, viruses, mold spores, algae and protozoa, for deodorizing and for retarding and/or controlling chemotaxis.

### SUMMARY OF THE INVENTION

Among the various aspects of the invention, therefore, may be noted the provision of an optically transparent or translucent compatible polymer blend that releases a concentration of chlorine dioxide or other gas sufficient to eliminate bacteria, fungi, molds and viruses; the provision of such a composition that can be melt processed; the provision of such a composition that will not react with chlorine dioxide or chlorite, can be easily processed at low temperature into film with good mechanical strength even after swelling with water, will form an IPN upon exposure to water thus permitting the water to access the interior of the film or molded object, will release chlorine dioxide or other relevant gas over an extended period when exposure to water mobilizes acidic groups in the hydrophobic polymer, and are compatible with sequestering agents which serve to retard ionic salt precipitation on surfaces.

In one embodiment, the present invention is directed to a compatible polymer blend for retarding bacterial, fungal and viral contamination and mold growth which comprises anions capable of reacting with hydronium ions to generate a gas; a hydrophilic polymer having a glass transition temperature of less than 100° C.; and either a hydrophobic polymer and an acid releasing agent, or an acid releasing hydrophobic polymer. The compatible polymer blend is substantially free of water and capable of generating and releasing the gas upon hydration of the acid releasing agent or the acid releasing hydrophobic polymer.

Another embodiment of the invention is directed to a compatible polymer blend for retarding bacterial, fungal and viral contamination and mold growth which comprises anions capable of reacting with hydronium ions to generate a gas; a hydrophilic polymer having the structure:

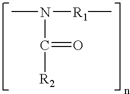

wherein R₁ is a substituted or unsubstituted alkylene group containing from 1 to 4 carbon atoms; R₂ is selected from a substituted or unsubstituted aryl group, a substituted or unsubstituted alkyl group containing from 1 to 6 carbon atoms; and wherein n is an integer which provides the polymer with a molecular weight of less than about 100,000 daltons; and either a hydrophobic polymer and an acid releasing agent, or an acid releasing hydrophobic polymer. The compatible polymer blend is substantially free of water and capable of generating and releasing the gas upon hydration of the acid releasing agent or the acid releasing hydrophobic polymer.

Another embodiment of the invention is directed to a process for preparing a compatible polymer blend having a melt temperature less than about 150° C., the process comprising forming a mixture or slurry of a liquid, anions, and a hydrophilic polyoxazoline polymer of the general formula:

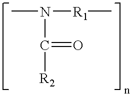

wherein R₁ is a substituted or unsubstituted alkylene group containing from 1 to 4 carbon atoms; R₂ is selected from a substituted or unsubstituted aryl group, a substituted or unsubstituted alkyl group containing from 1 to 6 carbon atoms; and wherein n is an integer which provides the polymer with a molecular weight of less than about 100,000 daltons; removing the liquid to form a glass; and melt blending the glass with either a hydrophobic polymer and an acid releasing agent, or an acid releasing hydrophobic polymer.

Yet another embodiment of the present invention is directed to a process for preparing a compatible polymer blend having a melt temperature less than about 150° C., the process comprising providing a mixture comprising anions and a hydrophilic polyoxazoline polymer; melt processing the mixture to form a glass; and melt blending the glass with either a hydrophobic polymer and an acid releasing agent, or an acid releasing hydrophobic polymer, wherein the hydrophilic polyoxazoline polymer has the formula:

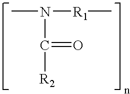

wherein R₁ is a substituted or unsubstituted alkylene group containing from 1 to 4 carbon atoms; R₂ is selected from a substituted or unsubstituted aryl group, a substituted or unsubstituted alkyl group containing from 1 to 6 carbon atoms; and wherein n is an integer which provides the polymer with a molecular weight of less than about 100,000 daltons.

Another embodiment of the invention is directed to a method of retarding bacterial, fungal, and viral contamination and growth of molds on a surface and/or deodorizing the surface comprising melt processing a compatible polymer blend of the invention to form an object or film; and exposing the surface of the object or film to moisture to release a gas from the compatible polymer blend into the atmosphere surrounding the surface to retard bacterial, fungal, and viral contamination and growth of molds on the surface and/or deodorize the surface.

Other aspects and advantages of the invention will be apparent from the following detailed description.

### DETAILED DESCRIPTION

In accordance with the present invention, it has been discovered that sustained release of a gas can be generated from an extrudable compatible polymer blend comprising a hydrophilic polymer, anions, and an acid releasing hydrophobic polymer, and/or a combination of a hydrophobic polymer and an acid releasing agent when the compatible polymer blend is exposed to moisture. Although gas releasing compositions are known, the compatible polymer blend is unique because it is optically transparent or translucent, may be melt extruded at temperatures as low as 90° C., and is a well dispersed blend of salts containing gas generating anions, hydrophobic and hydrophilic polymers. Furthermore, the hydrophobic polymers of the compatible polymer blend can release hydronium ions via hydration rather than hydrolysis, which avoids polymer chain cleavage and loss of structural integrity. The composition of the invention is advantageous because: the entire polymer blend is an active material (in contrast to known compositions in which the active portion is divided into layers); anion decomposition is inhibited; water transfer efficiency is enhanced; and a functional polymer is formed.

Unlike known optically transparent films which are formed by solvent based film casting, the compositions of the invention can be melt processed at temperatures of 90° C. or more. When the composition is applied to a substrate, the substrate can be clearly seen through the film formed on the substrate. If the composition, for example, is coated onto a container board box printed with graphics, the graphics remain clearly visible through the coating. Although the coating releases a gas, the coating does not alter the graphics or affect the color of the graphics. When the composition is extruded into a sterilizing packaging wrap or container that is used for product storage, product integrity can be clearly determined through the packaging. This is an especially important attribute when perishable consumer products such as food, cosmetics, pharmaceuticals or personal care products are packaged. When the composition is formed into sterilizing medical tubing, bandages, catheters, syringes, instruments, medical or biological waste storage media, and the like, visual monitoring of the medicament, medical device, or the patient are possible. The composition, therefore, allows visual inspection of a contained material while releasing a gas to sterilize, deodorize, and protect the material from contamination.

Gas releasing ions, including chlorite, are usually unstable in crystalline polymer solid matrices, and disproportionation to, for example, chlorate and chloride is favored at temperatures above about 160° C. High temperature chlorite decomposition may result in a finished product with insufficient chlorine dioxide generation capacity. Hence, the polymers of the present invention preferably should have a glass transition temperature (T_{g}) and melting temperature (Tₘ) less than about 160° C. Additionally, polymers should be capable of forming an interpenetrating network such that moisture may be absorbed into the hydrophilic polymer which may then extract chlorite ion from the dispersed chlorite containing salts and initiate acid release from the hydrophobic polymer or acid releasing agent. Further, the copolymers should not chemically react with the gas generating anion or gas. Finally, the composition should be transparent or translucent, and maintain the optical properties even upon water absorption, IPN formation and gas generation and release.

For purposes of the present invention, the term “compatible polymer blend” means a polymer blend where there is a sufficient interphase mixing and favorable interaction between the components so that the blend exhibits at least macroscopically uniform physical properties throughout its whole volume.

In one embodiment of the invention, the compatible polymer blend comprises a hydrophilic polymer, a salt containing anions capable of generating a gas, and either an acid releasing hydrophobic polymer or a hydrophobic polymer and an acid releasing agent. The gas is generated and released from the compatible polymer blend when water absorbed from the surrounding atmosphere causes the hydrophilic and hydrophobic polymers to separate into an interpenetrating network wherein the hydrophilic polymer comprises the anions and the hydrophobic polymer comprises the acid releasing agent or an acid releasing moiety. For purposes of the present invention, an interpenetrating network (“IPN”) is a material comprised of two or more phases in which at least one phase is topologically continuous from one free surface to another. The compositions of the present invention differ from two-phase compositions known in the art because the instant compositions are initially formed as a compatible blend polymer matrix comprising hydrophobic and hydrophilic copolymers. Upon exposure to ambient moisture, and if the relative humidity (“RH”) exceeds a threshold value, the polymer matrix is plasticized by water and forms an IPN, thereby permitting hydronium ion transport from the acid releasing groups to the gas-generating anions. Such a formulation is preferred for acidification of anions since the network efficiently allows moisture absorption and migration of generated hydronium ions from the acid releasing agent or moiety to the anions. Additionally, the presence of an interpenetrating hydrophobic polymer is useful for maintaining composite mechanical strength properties in the presence of a highly water plasticized hydrophilic polymer. In some cases small crystals may form in the hydrophobic phase which can physically crosslink the structure further increasing the mechanical strength. Conversely, if the RH does not exceed a threshold value, the polymer matrix will transmit water as a compatible blend. For example, when the anions are chlorite anions, the absorbed water diffuses and permits transfer of hydronium ions from the hydrophobic acid-releasing portion to the chlorite anion thereby forming chlorous acid with subsequent chlorine dioxide release. The gas diffuses out of the compatible polymer blend into the surrounding atmosphere in order to prevent growth of bacteria, molds, fungi and viruses on the coated material or formed object.

The inventive composition provides more efficient conversion to a gas, such as chlorine dioxide, than is provided by immiscible two-phase compositions known in the art because the IPN derived from an initially compatible blend with some interphase mixing provides greater surface to volume contact. Compositions that release at least about 0.3×10⁻⁶ to about 3.0×10⁻⁶ mole chlorine dioxide/cm² surface area for a period of at least 2 weeks, 3 weeks, 4 weeks, 5 weeks or even 6 weeks can be formulated by the processes of the present invention for a variety of end uses.

In one embodiment, the composition comprises from about 0.1 wt % to about 20 wt % of anions capable of generating a gas and counterions, 0 wt % to about 5 wt % of a base, about 15 wt % to about 60 wt % of a hydrophilic polymer, and about 30 wt % to 80 wt % of an acid releasing hydrophobic polymer and/or a combination of a hydrophobic polymer and an acid releasing agent. In another embodiment, the composition comprises from about 1 wt % to about 10 wt % of the anions and counterions, 0 wt % to about 3 wt % of the base, about 20% to 50% of the hydrophilic polymer, and about 30 wt % to 70 wt % of the acid-releasing hydrophobic polymer and/or a combination of a hydrophobic polymer and an acid releasing agent. In embodiments where an acid releasing agent is present, a weight ratio of hydrophobic polymer to acid releasing agent of from about 1 to about 25, from about 1 to about 4 or even from about 1 to about 1.5 is preferred.

Generally, any hydrophilic polymer that will support an electrolyte such as an inorganic anion is suitable for compositions of the invention. Preferably, the hydrophilic polymer is chemically compatible with the anion and does not promote significant gas generating anion instability or decomposition. The hydrophilic polymer preferably forms compatible blends with hydrophobic polymers of the present invention, the blends having melt processing temperatures (Tₘ) less than about 160° C. or even less than about 150° C., for example from about 90° C. to about 150° C., from about 90° C. to about 140° C., from about 90° C. to about 130° C., from about 90° C. to about 120° C. or even from about 90° C. to about 110° C. Melting temperature (Tₘ) is the temperature at which the structure of a crystalline polymer is destroyed to yield a melt processable material, and it is typically higher than T_{g}. Generally, the melt processing temperatures are achieved by the use of hydrophilic polymers having a sufficiently low T_{g} and Tₘ. For purposes of this invention, the glass transition temperature (T_{g}) is defined as the lowest temperature at which a non-crystalline polymer can be extruded or otherwise melt processed. The polymer is generally a hard and glassy material at temperatures less than T_{g}. A hydrophilic polymer with a T_{g} of less than about 100° C. is preferred. In one embodiment, an acceptable hydrophilic polymer T_{g} can be achieved by adding a plasticizer to lower its T_{g} below about 100° C. Alternatively, polymers may be selected that individually possess Tₘ values less than about 160° C., 150° C., 140° C., 130° C., 120° C. or even 110° C.

In an embodiment, the hydrophilic polymer has a molecular weight from about 1,000 and about 1,000,000 daltons, and will form a highly dispersed suspension with the salt containing the desired anions and a hydrophobic polymer. A highly dispersed suspension is defined as a mixture of components that each have a particle size of not more than about 1,000 angstroms, preferably not more than about 500 angstroms, and more preferably not more than about 100 angstroms as measured by microscopy or light scattering methods that are well known in the polymer art. A highly dispersed suspension of the present invention can also be a mixture comprising components that each have a particle size of not more than 2,000 angstroms when the index of refraction of each component of the mixture is the same or substantially similar. A highly dispersed suspension including components having any of the above particle sizes is optically transparent or translucent in appearance and visually appears to be a single phase mixture because its phase microstructure is of a diameter well below the wavelength of visible light. A highly dispersed suspension is optically transparent for purposes of the invention when at least about 80% of light, preferably at least about 90%, is transmitted through the suspension at the film thicknesses important for the application. The highly dispersed suspension does not scatter light and is stable to crystallization that would produce particles larger than 1000 angstroms. The particle size of the highly dispersed suspension is preferably small enough for the components to be uniformly dispersed.

The hydrophilic material preferably has a high hydrogen bonding density to enhance anion stability and can contain moieties including amines, amides, urethanes, alcohols, closed ring amides such as pyrrolidinone, or a compound containing amino, amido, anhydride or hydroxyl groups. The hydrophilic polymer most preferably includes amide, urethane, and anhydride groups. The anions generally do not react with the hydrophilic polymer but are surrounded, and stabilized, by hydrogen bonds contributed by the moieties within the hydrophilic polymer.

Hydrophilic polymers can include, for example, a polyoxazoline, poly n-vinyl pyrrolidinone (PNVP), a polyacrylamide, vinyl methyl ether and N-vinylacetamide. Hydrophilic polymers having a molecular weight of less than about 1,000,000 daltons, for example, from about 1,000 to about 100,000 daltons, or even from about 25,000 to about 75,000 daltons, are preferred.

Polyoxazolines are represented by the formula:

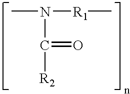

wherein R₁ is a substituted or unsubstituted alkylene group containing 1 to about 4 carbon atoms; R₂ is any hydrocarbon or substituted hydrocarbon that does not significantly decrease the water-solubility of the polymer; and n is an integer which provides the polymer with a molecular weight of less than about 1,000,000 daltons, preferably from about 1,000 to about 100,000 daltons, more preferably from about 25,000 to about 75,000 daltons. R₁ may be substituted with hydroxy, amide or polyether. R₁ is preferably methylene, ethylene, propylene, isopropylene or butylene. R₁ is most preferably ethylene. R₂ is preferably alkyl or aryl; R₂ may be substituted with hydroxy, amide or polyether. Preferably R₂ is methyl, ethyl, propyl, isopropyl, butyl, or isobutyl. Most preferably R₁ is ethylene and R₂ is ethyl.

Poly n-vinyl pyrrolidone (PNVP) polymers are represented by the formula:

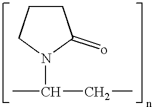

wherein n is preferably from about 10 to about 1000, more preferably from about 100 to about 900, and most preferably from about 200 to about 800.

Polyacrylamide polymers are represented by the formula:

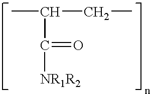

wherein R₁ and R₂ are independently hydrogen or any hydrocarbon or substituted hydrocarbon that does not significantly decrease the water-solubility of the polymer and wherein n is an integer which provides the polymer with a molecular weight of less than about 1,000,000 daltons, preferably from about 1,000 to about 100,000 daltons, more preferably from about 25,000 to about 75,000 daltons. For example, R₁ and R₂ can be a substituted or unsubstituted aryl group, or a substituted or unsubstituted alkyl group containing from 1 to about 6 carbon atoms. Preferably, R₁ and R₂ are independently hydrogen, aryl or alkyl. More preferably, R₁ and R₂ are independently hydrogen or C₁₋₄ alkyl. Even more preferably R₁ and R₂ are independently hydrogen or methyl.

Any hydrophobic polymer that will form compatible blends with hydrophilic polymers, is compatible with the gas generating anions, and has a T_{g} and Tₘ value adequate for melt processing in the presence of the anions is acceptable for the purposes of the present invention. Generally any hydrophobic polymer capable of a hydrogen bonding interaction with the hydrophilic polymer will form compatible polymer blends. Without being bound to any theory, experimental evidence to date indicates that transparent, compatible polymer blend are produced when the hydrogen-contributing hydrophilic polymers form bonds with hydrophobic polymers containing a threshold number of hydrogen bonding or compatabilizing groups. The groups include, but are not limited to, hydroxyl, amide, anhydride, carboxylic acid, nitrile, ester, acid salts, urethanes, fluoride, and chloride.

Hydrophobic polymers and copolymers acceptable for purposes of the present invention include a large number of alkyl or aromatic based polymers and may comprise substituted or unsubstituted polyalkylene acrylic acids (e.g., polyethylene acrylic acid (PEAA)), and their partially neutralized salts, alkylene-methacrylic acids (e.g., ethylene-methacrylic acid (EMAA)), and their partially neutralized salts, phenoxy resins, monoalkyl itaconic acids, alkylene-vinyl alcohols (e.g., ethylene vinyl alcohol (EVA)), alkylene acrylic acids (e.g., ethylene-acrylic acid (EAA)), alkyl-vinyl alcohol and polyalkylene blends, alkyl-vinyl alcohol and vinyl alcohol blends, vinylacetate (VAC) and vinyl alcohol blends, cellulose acetates, aromatic polyimides, vinylidine fluoride, polyacrylic acids, poly(vinylsulfonic acid), poly(styrenesulfonic acid), polyalkylene oxides (e.g., polypropylene oxide), polystyrenes, vinyl chlorides, vinyl acetates and salts thereof. Preferably the hydrophobic polymer has a molecular weight from about 1,000 to about 1,000,000 daltons, and more preferably from about 10,000 to about 100,000 daltons.

In one embodiment, the hydrophobic polymer comprises an acid releasing moiety. The acid releasing hydrophobic polymer can release a hydronium ion by a hydration mechanism upon exposure to moisture resulting in protonation of the anion with subsequent release of gas. Hydrophobic polymer acid releasing moieties of the present invention are preferably present as side groups rather than as an integral structural component of the polymer backbone chain. When an acid releasing moiety is present as an integral structural component of the polymer chain, the moiety must first be hydrolyzed before hydration and acid release can occur. Hydration of the hydrolyzed moiety results in polymer chain cleavage and the structural integrity of the polymer is compromised. Because the acid releasing moieties of the hydrophobic polymers of the present invention are present as side groups, hydrolysis of the polymer backbone does not occur and polymer structural integrity and mechanical properties of the polymer are maintained Hydrophobic polymers comprising carboxylic acid moieties are most preferred. Acid releasing hydrophobic polymers thus enable compositions to be made without the inclusion of a separate acid releasing component. Such compositions provide several advantages over compositions containing an added acid releasing component. First, material cost may be reduced. Second, greater anion loading can be achieved when the separate acid releasing component is eliminated. And third, acid releasing agents may cause translucent or cloudy compositions because they are either formulated as a powder or may precipitate upon IPN formation.

Preferred alkylene-methacrylic acid copolymers, alkylene-acrylic acid copolymers, and copolymers of their respective esters have the formula:

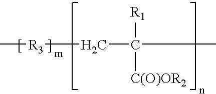

wherein R₁ is independently selected from hydrogen and substituted or unsubstituted lower alkyl, R₂ is independently selected from hydrogen or substituted or unsubstituted lower alkyl, and R₃ is ethylene or propylene. The ratio of m to n is from 99:1 to 1:99, 50:1 to 1:50, 25:1 to 1:25, 25:1 to 1:10, 25:1 to 1:1, 20:1 to 1:1, 15:1 to 1:1, 20:1 to 5:1, 15:1 to 5:1, 10:1 to 1:1 or even 8:1 to 2:1. Preferably R₁ is independently selected from hydrogen, methyl or ethyl, R₂ is independently selected from hydrogen, methyl, ethyl, n-propyl or isopropyl and R₃ is ethylene. R₂ may also comprise a salt forming cation such as an alkali metal, zinc, or ammonia. In one embodiment, R₁ is independently hydrogen or methyl, R₂ is independently methyl or ethyl and R₃ is ethylene. In another embodiment, R₁ is hydrogen, R₂ is hydrogen and R₃ is ethylene.

Preferred monoalkyl itaconic acid and monoalkyl itaconate copolymers have the formula:

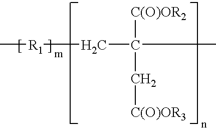

wherein R₁ is a substituted or unsubstituted lower alkylene and R₂ and R₃ are independently hydrogen or substituted or unsubstituted lower alkyl; more preferably R₁ is ethylene or propylene, and R₂ and R₃ are independently hydrogen, methyl, ethyl, n-propyl, iso-propyl, n-butyl, iso-butyl or The ratio of m to n is from 99:1 to 1:99, 50:1 to 1:50, 25:1 to 1:25, 25:1 to 1:10, 25:1 to 1:1, 20:1 to 1:1, 15:1 to 1:1, 20:1 to 5:1, 15:1 to 5:1, 10:1 to 1:1 or even 8:1 to 2:1. R₂ or R₃ may also independently comprise a salt forming cation such as an alkali metal, zinc, or ammonia.

Weight ratios of total hydrophilic polymer to hydrophobic polymer can suitably be about 85:15, about 80:20, about 75:25, about 70:30, about 65:35, about 60:40, about 55:45, about 50:50, about 45:55, about 40:60, about 35:65, about 30:70, about 25:75, about 20:80 or about 15:85, based upon the total weight of hydrophilic and hydrophobic polymers within the composition of the invention.

The compositions contain anions which react with hydronium ions to generate a gas. The anions are generally provided by salts of the anions and a counterion. Suitable salts include an alkali metal chlorite, an alkaline-earth metal chlorite, a chlorite salt of a transition metal ion, a protonated primary, secondary or tertiary amine, or a quaternary amine, an alkali metal bisulfite, an alkaline-earth metal bisulfite, a bisulfite salt of a transition metal ion, a protonated primary, secondary or tertiary amine, or a quaternary amine, an alkali metal sulfite, an alkaline-earth metal sulfite, a sulfite salt of a transition metal ion, a protonated primary, secondary or tertiary amine, or a quaternary amine, an alkali metal bicarbonate, an alkaline-earth metal bicarbonate, a bicarbonate salt of a transition metal ion, a protonated primary, secondary or tertiary amine, or a quaternary amine, an alkali metal carbonate, an alkaline-earth metal carbonate, a carbonate salt of a transition metal ion, a protonated primary, secondary or tertiary amine, or a quaternary amine, Preferred salts include sodium, potassium, calcium, lithium or ammonium salts of a chlorite, bisulfite, sulfite, bicarbonate, or carbonate. Commercially available forms of chlorite and other salts suitable for use, such as Textone® (Vulcan Corp.), can contain additional salts and additives such as tin compounds to catalyze conversion to a gas.

Other forms of chlorite such as Microsphere® powder or a silicate-chlorite solid solution, as disclosed, for example in U.S. Pat. Nos. 6,605,304 and 6,277,408 (to Wellinghoff), incorporated herein by reference, may be incorporated into compositions of the invention. Microsphere® powders have relatively low chlorite loading, hence that material is suitable for compositions providing slow chlorine dioxide release. Microsphere® powder and silicate-chlorite particle size is preferably from about 1 to about 10 microns.

Compositions of the invention may also be blended with electromagnetic energy activated gas releasing compositions as described in U.S. patent application Ser. No. 09/448,927 and PCT Publication No. WO 00/69775, incorporated by reference herein, or combined in multilayer films to provide a moisture and/or electromagnetic energy activated composition effective for applications as described herein.

Chlorite sources that are generally stable at processing temperatures in excess of about 100° C., thereby allowing for processing at relatively high temperatures, are preferred. Preferred chlorite sources that can be incorporated into the composition of the present invention include sodium chlorite, potassium chlorite, calcium chlorite, Microsphere® powder and sodium chlorite powder, as is available commercially under the trademark Textone®. Since the chlorite content of such powders is high, compositions of the invention including such powders are active chlorine dioxide emitters. Moreover, in some applications micronized sodium chlorite based glasses are preferred over solubilized or nanoparticle sodium chlorite glasses because the low surface to volume ratio of the chlorite particulate retards reaction with the hydrophobic acid releasing groups during melt processing. However, the benefits of larger particle size chlorite must be balanced against the increased light scattering and film translucency that result from the incorporation of the large particles.

Maximum chlorine dioxide release from a composition can be achieved by stabilizing the chlorite anion. Water solutions of chlorite normally are quite basic and the long term stability of chlorite anion in these solutions depends on the pH remaining basic. Even low concentrations of protons will result in the formation of small amounts of chlorous acid which will disproportionate to chlorine dioxide. In one embodiment of the invention, a chlorite anion source, the hydrophilic polymer and a base are prepared from solution (for example, by casting) to produce a transparent, brittle glass containing the inorganic components dispersed molecularly or as nanoparticles. Based on experimental evidence to date, and without being bound to any theory, it is believed that during the evaporation stage of the preparation process, increasing amounts of unstable HClO₂ form as the strong complexation of ClO₂ by aqueous or organic solvents is replaced by the weaker amide chelation. Hydroxide ion contributed by the base disfavors the formation of chlorous acid, thus enhancing the stability of the formed glass. Preferably the molar ratio of chlorite anions to hydroxide anions is from about 1:2 to about 10:1, more preferably from about 2:1 to about 10:1.

In general, any base can be incorporated in the composition. Suitable bases include, but are not limited to, an alkali metal bicarbonate such as lithium, sodium, or potassium bicarbonate, an alkali metal carbonate such as lithium, sodium or potassium carbonate, an alkaline-earth metal bicarbonate, an alkaline-earth metal carbonate such as magnesium or calcium carbonate, a bicarbonate salt of a transition metal ion, a protonated primary, secondary or tertiary amine, or a quaternary amine such as ammonium bicarbonate, a carbonate salt of a transition metal ion, a protonated primary, secondary or tertiary amine, or a quaternary amine, an alkali metal hydroxide such as lithium, sodium or potassium hydroxide, an alkaline-earth metal hydroxide such as calcium or magnesium hydroxide, a hydroxide salt of a transition metal ion, a protonated primary, secondary or tertiary amine, or a quaternary amine such as ammonium hydroxide, an alkali metal phosphate such as dibasic or tribasic phosphate salts, an alkaline-earth metal phosphate such as bicalcium or tricalcium phosphate, a phosphate salt of a transition metal ion, a protonated primary, secondary or tertiary amine, or a quaternary amine, Preferred bases include sodium hydroxide, potassium hydroxide and ammonium hydroxide. Sodium hydroxide is most preferred.

Hydronium ions can be provided by hydrophobic polymers comprising an acid releasing moiety or by an acid releasing agent that is incorporated in the compositions. Moisture activated, acid releasing agents as disclosed, for example, in U.S. Pat. Nos. 6,277,408 and 6,046,243 (both to Wellinghoff), both of which are incorporated herein, may optionally be added to the polymer blend to permit protonation of the anion with subsequent release of gas. Any acid releasing agent that is capable of being incorporated into an inventive composition comprising hydrophilic and hydrophobic polymers and anions is acceptable for purposes of the present invention. Preferably, the acid releasing agent does not react with the composition components in the absence of moisture, and does not exude or extract into the environment. Suitable acid releasing agents include inorganic salts, carboxylic acids, esters, acid anhydrides, acyl halides, phosphoric acid, phosphate esters, trialkylsilyl phosphate esters, dialkyl phosphates, sulfonic acid, sulfonic acid esters, sulfonic acid chlorides, phosphosilicates, phosphosilicic anhydrides, carboxylates of poly α-hydroxy alcohols such as sorbitan monostearate or sorbitol monostearate, and phosphosiloxanes.

Preferred acid anhydride releasing agents include organic acid anhydrides, mixed organic acid anhydrides, homopolymers of an organic acid anhydride or a mixed inorganic acid anhydride, and copolymers of an organic acid anhydride or a mixed inorganic acid anhydride with a monomer containing a double bond. The presence of an anhydride increases the acidity and the metal ion sequestering capability of the composition. Metal ion sequestering potential helps alleviate surface metal salt precipitation that potentially occurs when the compositions are hydrated. Preferred mixed inorganic acid anhydrides contain a phosphorus-oxygen-silicon bond. Preferred anhydrides include copolymers containing maleic anhydride, methacrylic anhydride, acetic anhydride, propionic anhydride, or succinic anhydride. Copolymers of acid anhydrides and esters of lactic or glycolic acids can provide a rapid initial gas release rate followed by a slow release rate.

Inorganic acid releasing agents, such as polyphosphates, are also preferred acid releasing agents because they form odorless powders generally having greater gas release efficiency as compared to powders containing an organic acid releasing agent. Suitable inorganic acid releasing agents include tetraalkyl ammonium polyphosphates, monobasic potassium phosphate, potassium polymetaphosphate, sodium metaphosphates, borophosphates, aluminophosphates, silicophosphates, sodium polyphosphates such as sodium tripolyphosphate, potassium tripolyphosphate, sodium-potassium phosphate, and salts containing hydrolyzable metal cations such as zinc.

Linear or star like oligomers (e.g., a micelle-like molecule with a lipid wall and a P—O—Si core), such as a phosphosilicic anhydride that is the reaction product of a phosphoric acid ester of a C₄ to C₂₇ organic compound and a silicate ester, are preferred acid releasing agents because they can be melt processed with the option of being crosslinked after processing to provide film stability. Preferred phosphosilicic anhydrides of esters comprise a carboxylic acid ester of a polyhydric alcohol and a C₄ to C₂₇ hydrocarbon singly or multiply substituted with hydroxy, alkyl, alkenyl, or esters thereof. Preferred phosphosilicic anhydrides of polyol based esters include alkylene glycol fatty acid ester acid releasing waxes such as propylene glycol monostearate acid releasing wax. A preferred phosphosilicic anhydride of a glycerol based ester is LPOSI, or glycerol monostearate acid releasing wax. See U.S. Pat. No. 5,631,300 (to Wellinghoff), incorporated by reference herein.

Ester modified copolymers such as, for example, ethylene methacrylic, ethylene acrylate and ethylene vinyl acetate may be added as diluents. The ester groups form hydrogen bonds with hydrophilic polymer amide groups to promote the formation of a compatible blend. These additives enable a wider range of hydrophilic polymers to be used, promote the formation of compatible polymer blends, and permit greater loading of gas forming anions.

Plasticizers may be added to the compositions of the present invention to suppress T_{g}, suppress Tₘ, lower viscosity, act as a surfactant to disperse the acid releasing agent, influence moisture uptake rate, and/or form a supple and flexible film. Plasticizers preferably form a compatible blend with the hydrophilic and hydrophobic polymers. Plasticizers such as alkylene glycols (for example, PEG) do not form compatible blends with the hydrophilic and hydrophobic polymers of the present invention and are generally not preferred. In one embodiment, melt processing properties of the composition may be modified by the addition of low molecular weight PEOX or other low molecular weight amides. The additives may alter the composite T_{g}, water solubility, mechanical properties, and rheological properties including viscosity and flow characteristics to allow low temperature processing and prevent embrittlement and cracking. Generally up to about 30 weight percent of a plasticizer may be added. A glassy polymer can be softened to increase mobility by adding at least about 10% by weight, preferably from about 10 to about 30% by weight of a plasticizer to lower glass transition temperature below the reaction temperature. Generally any plasticizer that will plasticize polyamide and that is not easily oxidized is acceptable. Preferred phthalate plasticizers include dibutyl phthalate, and dioctly phthalate. Preferred PEOX and amide plasticizers preferably have a molecular weight of about 5000 daltons. Suitable low molecular weight amide plasticizers are well known in the polymer art and may include monomeric or oligomeric amides such as succinamide, formamide, N-methyl formamide, N-ethylformamide, N-methylacetamide, N-ethylacetamide, isopropylacrylamide-acrylamide and amido substituted alkylene oxides. Formamide and N-methyl formamide are toxic and would not be preferred in applications involving human contact. Other amides that can be used as plasticizers for the acid releasing polymer of the invention include H₂NC(O) (CH₂CH₂O)ₙCH₂CH₂C(O)NH₂ wherein n is 1 to 10, H₂NC(O)(CH₂CH₂O)ₙCH((OCH₂CH₂)ₘC(O)NH₂)₂ wherein n is 1 to 5 and m is 1 to 5, and N(CH₂CH₂O)ₙCH₂CH₂ (O)NH₂)₃ wherein n is 1 to 10.

Other polymers can be added to the composition to improve or optimize properties such as, for example, strength, toughness, flexibility and/or gas releasing characteristics. In one embodiment, alkylene-vinyl alcohol copolymers that may be introduced into the blend have the formula:

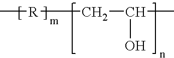

wherein R is a substituted or unsubstituted lower alkylene, preferably ethylene or propylene. The ratio of m to n is from 99:1 to 1:99, 50:1 to 1:50, 25:1 to 1:25, 25:1 to 1:10, 25:1 to 1:1, 20:1 to 1:1, 15:1 to 1:1, 20:1 to 5:1, 15:1 to 5:1, 10:1 to 1:1 or even 8:1 to 2:1.

In another embodiment, aromatic polyimide additives that may be introduced into the blend have the formula:

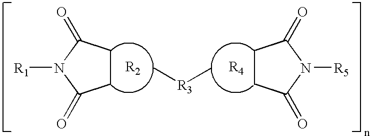

wherein R₁ and R₅ are independently hydrogen, alkyl, alkenyl, alkanoyl, carboxyalkyl, alkoxy, alkoxycarbonyl, alkylaminoalkyl, alkylcarbonyl, alkylcarbonylalkyl, aryl, alkylsulfinyl, aryl, acyl, carboxy, carbonyl, cycloalkenyl, cycloalkyl, ester, haloalkyl, heteroaryl, heterocyclo, hydroxyalkyl, sulfamyl, sulfonamidyl, sulfonyl, alkylsulfonyl, arylsulfonyl or oxo; and R₃ is independently alkylene, alkenylene, alkanoylene, carboxyalkylene, alkenoxy, alkenoxycarbonyl, alkenylaminoalkyl, alkenylcarbonyl, alkenylcarbonylalkyl, alkenylsulfinyl, aryl, acyl, carboxy, carbonyl, cycloalkenyl, cycloalkyl, ester, haloalkenyl, heteroaryl, heterocyclo, hydroxyalkenyl, sulfamyl, sulfonamidyl, sulfonyl, alkylsulfonyl, arylsulfonyl or oxo; R₂ and R₄ are independently cyclohexyl, aryl, cycloalkenyl, cycloalkyl, heteroaryl or heterocyclo; preferably R₁ is arylene or alkene, R₂ comprises aryl, R₃ is alkene, R₄ is aryl and R₅ is arylene or alkene; most preferably R₁ is alkene, R₂ is phenyl, R₃ is methylene, R₄ is phenyl and R₅ is alkene.

A moisture scavenger, such as sodium sulfate, calcium sulfate, silica gel, alumina, zeolites, and calcium chloride can be added to the composition to prevent premature hydrolysis of the acid releasing hydrophobic polymer or acid releasing agent. Conversely, humectants can be added to render the composition more hydrophilic and increase the rate of hydrolysis of the acid releasing hydrophobic polymer or acid releasing agent. Conventional film forming additives can also be added to the composition as needed. Such additives include crosslinking agents, flame retardants, emulsifiers, UV stabilizers, slip agents, blocking agents, and compatibilizers, lubricants, antioxidants, colorants and dyes. These additives must be hydrophilic and soluble within the composition if the composition is to be optically transparent or translucent.

The extruded compatible polymer blends of the present invention are hygroscopic and are significantly plasticized by water, and upon exposure to water will form an IPN. In general, IPNs of the present invention are continuous and comprise water rich and water lean phases formed for the acidification of anions to produce a gas. Water can then diffuse into the interior of the composite to permit proton transport from the hydrophobic polymer acid releasing groups or the acid releasing agent to the gas generating anions.

Under one theory, and without being bound to any particular theory, it is believed that the IPN is formed by water exposure generating a continuous phase rich in water and hydrophilic polymer within a continuous or semi-continuous phase rich in hydrophobic polymer. Formed water channeling is partially a function of the swelling capability of the hydrophilic polymer counterbalanced by the bonding forces between hydrophilic and hydrophobic polymer functional groups. Hence a composition with a large channel size generally comprises a highly swollen hydrophilic phase coupled with low bonding strength between the formed phases. Conversely, small channel sizes generally result from a combination of a minimally swollen hydrophilic phase strongly bonded with the hydrophobic phase. Other factors including solvent systems, anion content, extrusion temperature and ambient humidity can affect formed channel morphology.

Under another theory of IPN formation, the miscibility of polymer mixtures is governed by the thermodynamics of mixing. If the Gibbs free energy of mixing at a given temperature is negative then the polymer blend on the molecular level is more stable than a macroscopic mixture of individual components and a homogeneous mixture results. A change in free energy may occur if the stable homogeneous mixture of two polymeric components of the present invention is exposed to water. If the free energy change creates an unstable system, the blend can lower its total free energy and reach a stable state by demixing into two phases in a process termed spinodal decomposition. Such a phase separation can form an interpenetrating structure of the polymers.

In yet another theory of IPN formation, water induces the nucleation and growth of the polymer lean phase. Introduction of water into the compatible polymer blend causes a systemic free energy change. The blend reaches a new thermodynamic stability by demixing into two phases by nucleation and growth of the polymer lean phase thereby forming IPNs. It is believed that the polymer, at a critical polymer concentration, precipitates in discrete microdomains around a core structure which may be the initial portion of a new phase. The nucleation sites then grow into larger particles which may combine into an IPN. Factors such as polymer concentration, RH or temperature may cause microdomain nucleation to initiate at different times and proceed at different rates resulting in formed IPNs having hydrophilic channels exhibiting a variety of shapes and sizes.

In the present invention, it has been discovered that if the water source is ambient water vapor, then a threshold relative humidity (RH) is required to form an IPN. The threshold RH varies with a number of variables including, but not limited to: the hydrophobic and hydrophilic polymer constituent composition, including monomer or copolymer structure and molecular weight, and their respective concentrations; temperature; and anion, stabilizing base, plasticizer, moisture scavenger and humectant composition and loading. Moreover, the porosity of formed interpenetrating networks is influenced by these variables. For example, H. Chae Park et al. have found that the size of hydrophilic phase channels formed by exposing membranes composed of polysulfone and 1-methyl-2-pyrrolidone to water vapor is influenced by both RH and polymer concentration. It was found that channel size and RH, as well as pore size and polymer concentration are inversely related. Thus channel size increases with decreasing RH for a given polymer concentration, and channel size decreases with increasing polymer concentration at a given RH. See H. Chae Park et al., Journal of Membrane Science 156 (1999) 169-178.

Depending upon the ambient RH, the polymer matrix will either transmit water as a compatible blend or will form an IPN comprising the hydrophobic polymer and the hydrophilic polymer. Upon exposure to RH exceeding a threshold value, the polymer blend is plasticized by water and forms an IPN, thereby permitting hydronium ion transport from the acid releasing groups to the gas-generating anions. The gas is released from these blends over a period of days to weeks. Conversely, exposure to RH below the threshold value gives compatible blend water transmission with subsequent retarded gas release. The water transmission rate, and thus the gas release profile, can be adjusted for a wide range of conditions by altering both composition and ambient humidity.

The presence of an interpenetrating hydrophobic polymer is useful for maintaining mechanical properties in the presence of the highly water plasticized hydrophilic polymer, and other additives such as plasticizers. The hydrophobic polymer provides a matrix structure to maintain the structural integrity and prevent deformation of inventive objects during the course of intended use. This is an important property for objects such as, for example, tubing which may be subjected to pressure, medical devices requiring close tolerances, and vials, tubes, bottles and the like which may contain biological or hazardous materials.

The components of the composition are substantially free of water to avoid significant release of gas prior to use of the composition. For purposes of the present invention, the composition is substantially free of water if the amount of water in the composition does not provide a pathway for transmission of hydronium ions from the acid releasing hydrophobic polymer or acid releasing agent to the gas generating anions. Generally, the components of the composition can include up to a total of about 1.0% by weight water without providing such a pathway for transmission of hydronium ions. Preferably, each component contains less than about 0.1% by weight water, and, more preferably, from about 0.01% to about 0.1% by weight water. Insubstantial amounts of water can hydrolyze a portion of the acid releasing hydrophobic polymer or acid releasing agent to produce acid and hydronium ions within the composition. The hydronium ions, however, do not diffuse to the gas generating anions until enough free water is present for transport of hydronium ions.

Compatible polymer blends of the invention can be produced by a variety of methods. In one embodiment of the invention, a solution containing an anion source, a base, and a compatible hydrophilic polymer is prepared and solvent such as water, methanol or ethanol is then removed to produce a transparent, compatible phase glass or one containing nanomeric crystals of the salt. The glass serves as an organic based concentrate material that can be subsequently melt blended with suitable hydrophobic polymers and additives such as, for example, acid releasing agents. In one such embodiment, the source of anions is a commercial source of chlorite such as Textone, the base is sodium hydroxide, and the hydrophilic polymer is polyoxazoline or poly n-vinyl pyrrolidinone. Preferably chlorite is cast up to about 20% by weight active salts, more preferably up to about 15%, and most preferably up to about 10% by weight. A threshold amount of base is preferred to stabilize the gas generating anions and assure that the anions survive the casting process from the solvent. Preferably the weight percent ratio of base to gas generating anions such as chlorite is from about 1:2 to about 1:10, and most preferably about 1:4. The glasses may be true solid solutions of the anionic material in hydrophilic polymer, or may be fine dispersions of nanoparticles. Because anionic material particle size is small, glass based composites advantageously maximize optical clarity and can be used to obtain optically clear films and melt processed blends.

In one embodiment, the concentrate material is formed by rapid evaporation of a solution containing the anion source, base and hydrophilic polymer. In another embodiment, a dry powder suitable for blending can be produced in a spray dryer by limiting the exit temperature of the spray dryer to less than the powder T_{g}. In yet another embodiment, aqueous or solvent solutions may be cast in large area pans followed by vacuum drying at temperatures from about 50 to about 80° C. to produce brittle, clear glasses which can subsequently be powdered. Preferably, water-plasticized mixtures of anions, base and hydrophilic polymer (e.g., sodium chlorite, sodium hydroxide and PEOX polymer) are fused and extruded at a temperature from about 50 to about 80° C. through a slit die and the film is then thinned by drawing out on a moving release film substrate. The film is then air dried above the T_{g} of the unplasticized hydrophilic polymer (e.g., PEOX) to assure maintenance of film ductility and high water diffusion rates, and then cooled on rollers to below T_{g}. The brittle solid is then detached from the underlying release film and ground to a concentrate powder.

The glass or concentrate powder can be melt blended with hydrophobic polymers to produce a melt processable compatible polymer blend capable of controlled release of a gas. In one embodiment, the glass or concentrate powder is melt blended with hydrophobic polymer (e.g., polyethylene acrylic acid polymers (PEAA)) in hydrophilic polymer (e.g., PEOX) to hydrophobic polymer ratios of about 35:65 to about 45:55. In another embodiment, a PEOX containing glass having an average molecular weight of about 50,000 daltons is melt blended with PEAA having an average molecular weight of about 20,000 daltons to produce a compatible polymer blend characterized by limited light scattering, thermodynamic stability and capability of controlled release of chlorine dioxide gas. The extruded compatible polymer blends are hygroscopic, significantly plasticized by water and can form an IPN when exposed to water. Water can thus diffuse to the interior of the composition to permit proton transport from the PEAA carboxylate groups to the chlorite anion forming chlorous acid and thereby releasing chlorine dioxide.

In another embodiment, an acid releasing agent can be solubilized in the hydrophilic polymer with the anions and then be melt processed with the hydrophobic polymer to form transparent glasses. Examples of suitable acid releasing agents for this embodiment are inorganic compounds including sodium polyphosphate (NaPO₃) tetraalkyl ammonium polyphosphates, monobasic potassium phosphate (KH₂PO₄), potassium polymetaphosphate ((KPO₃)ₓ wherein x ranges from 3 to 50), sodium metaphosphates, borophosphates, aluminophosphates, silicophosphates, sodium polyphosphates such as sodium tripolyphosphate, potassium tripolyphosphate (K₅P₃O₁₀), sodium-potassium phosphate (NaKHPO₄.7H₂O), and salts containing hydrolyzable metal cations such as zinc. Suitable sodium metaphosphates have the formula (NaPO₃)ₙ wherein n is 3 to 10 for cyclic molecules and n is 3 to 50 for polyphosphate chains. Generally, the inorganic acid releasing agents may be formulated at solid weight percentages of up to about 15% by weight.

In a further embodiment, a hydrophobic polymer can be co-extruded with an anion salt-loaded hydrophilic polymer in order to decrease melt viscosity and improve composite mechanical properties. If the hydrophobic polymer is not acid releasing then an acid releasing agent can be added prior to melt blending. Optionally, stabilizers, plasticizers, surfactants, humectants or desiccants can be added. Preferred stabilizers include alkali hydroxide, and a preferred plasticizer is polyethylene. Inclusion of active surfactants such as octadecyl succinic anhydride enables greater concentrations of plasticizer such as polyethylene to be effectively incorporated.

In another embodiment, a finely powdered anion salt source is mixed with one or more hydrophilic polymers and one or more hydrophobic polymers, and melt processed at temperatures from about 90 to about 150° C. In one process option, a dry blend comprising the anion salt source, one or more hydrophilic polymers and one or more hydrophobic polymers is formed that is subsequently processed by melting, such as by melt extrusion. If the hydrophobic polymer is not acid releasing then an acid releasing agent can be added prior to melt processing. Optionally, stabilizers, plasticizers, surfactants, humectants or desiccants can be added. Preferred stabilizers include alkali hydroxide, and a preferred plasticizer is polyethylene. Chlorite salts are the preferred anion source and may be either in pure form or a neat chlorite from a source such as Textone®.

In addition to formation of functional melt processable compatible polymer blends, the compositions of the present invention can be applied as a film by using hot melt, dip coat, spray coat, curtain coat, dry wax, wet wax, coextrusion and lamination methods known to those skilled in the art.

In one embodiment for the industrial scale preparation of polymeric articles and films from the compatible polymer blends of the present invention, the hot molten polymer is extruded as a strand into a water quench bath where the polymer solidifies. The solidified polyolefin strand is then typically pellitized, subjected to size classification to remove off-sized pellets, and collected and packaged, for example in moisture vapor barrier packaging. Pellets can then be further processed by methods known in the art, such as by extrusion, to prepare polymeric articles and films of the present invention.

The compatible polymer blends of the present invention are activated by hydration and therefore are preferably shielded from water during the water quench. In one embodiment, the polymer blends are shielded from hydration by a wax surface coating. In this embodiment, an incompatible wax is admixed with the compatible polymer blend prior to extrusion. In this context “incompatible” means that the wax has only limited solubility with the polymer blend. During film extrusion, the wax migrates throughout the polymer blend to the surface thereof in a controlled manner (i.e., the wax “blooms” at the polymer blend surface) thereby providing a moisture barrier during subsequent water quenching. Under one theory, and without being bound to any particular theory, it is believed that the wax molecules migrate more freely in the admixture in the molten state (i.e., during extrusion) than the polymer molecules because of the lower molecular weight of the wax as compared to the polymers, the difference in polarity between the wax and polymers, the level of saturation of the wax hydrocarbon chain, the conformation and spatial structure of the polymer molecules, or combinations thereof. The rate of wax diffusion to the surface of the formed polymer film or article is termed the “bloom rate.” The wax acts as a barrier shielding or partially shielding physical contact between water and the polymer surface. The wax predominantly stays on the surface of the pellet and upon further processing acts as a slip and release agent because smoothness of the surface of the formed compatible polymer blend lowers its coefficient of friction

Both natural and synthetic waxes can be employed, including petroleum waxes such as olefinic waxes (predominately straight-chain saturated hydrocarbons) and microcrystalline wax (predominately cyclic saturated hydrocarbons with isoparaffins), vegetable waxes (e.g., carnauba), mineral waxes, and animal waxes (e.g., spermaceti) waxes. Olefinic waxes and oils are preferred. By “olefinic wax or oil” is meant hydrocarbons, or mixtures of hydrocarbons, having the general formula CₙH₂₊₂. Exemplary olefinic waxes or oils include paraffin waxes, nonoxidized polyethylene waxes, and liquid and solid hydrocarbons such as paraffin oil. An example of a suitable wax is Sasol Enhance 1585 wax having a molecular weight of about 1000 daltons available from Sasol Wax (South Africa).

The wax has a lower molecular weight than the polymers, preferably from about 500 to about 9000 daltons, more preferably from about 500 to about 6000 daltons, and most preferably from about 500 to about 3000 daltons. The wax melting point is preferably from about 50° C. to about 150° C., depending upon the chain length. The waxes preferably have a Brookfield viscosity in the range of from about 50 to about 700 cps @ 140° C. and a density in the range of from about 0.85 to about 0.95. The wax is typically blended with compatible polymer blends of the present invention in an amount of from about 0.1 wt % to about 8 wt % based on the total weight of the compatible polymer blend, preferably from about 1 wt % to about 6 wt %, and most preferably from about 3.5 wt % to about 5 wt %.

The wax and the compatible polymer blend can be admixed in various ways. In a first embodiment, the two components can be separately fed in two streams into the feed throat of an extruder. In another embodiment, the wax, anions, hydrophobic polymer and hydrophilic polymer can be premixed to form a melt blend. Suitable blending devices include twin screw extruders, kneaders or blenders (e.g., a Henschel mixer). In another embodiment, the wax can be added to a solution containing a solvent such as water, methanol or ethanol, an anion source, and a compatible hydrophilic polymer from which a glass is formed by solvent evaporation. In one embodiment, blending devices and packaging containers are purged with nitrogen to provide a low moisture environment.

Suitable melt extrusion methods used to form films, tubes or other objects from the composition of the present invention include extrusion molding, injection molding, compression molding, blow molding and other melt processing methods known in the art. In extrusion molding, polymer pellets are fed through a heating element to raise the temperature above T_{g}, and Tₘ and the resulting plasticized polymer is then forced through a die to create an object of desired shape and size. Extrusion molding is generally used to produce sutures, tubing and catheters. Optionally however, a gas can be blown into the extruder to form polymer bags and films from the plasticized polymer. Injection molding involves heating polymer powder or pellets above T_{g}, and in some cases above Tₘ, pressurized transfer to a mold, and cooling the formed polymer in the mold to a temperature below T_{g} or Tₘ. In compression molding, solid polymer is placed in a mold section, the mold chamber is sealed with the other section, pressure and heat are applied, and the softened polymer flows to fill the mold. The formed polymer object is then cooled and removed from the mold. Injection molding and compression molding are generally used to manufacture syringes, medical instrument and device parts, food-ware and the like. Finally, blow molding entails extrusion of a plasticized polymer tube into a mold and blowing up the tube to fill the mold. This method is generally used to produce relatively large containers such as bottles, jugs, carboys and drums.

The compositions of the present invention can also be used in forming a multilayered composite wherein the gas-generating compatible polymer blend of the invention (second layer) is sandwiched between films (first and third layers) which control the permeation of water vapor which is necessary for the release of the gas. The compatible polymer blends can then be made to exhibit different release profiles by controlling the rate of moisture ingress into the water-soluble layer to control gas release from the multilayered composite when activated by moisture. Further, the surrounding films may also impart mechanical strength to the composite that could not be achieved by the compatible polymer blend layer alone. Composites of the invention may be separately extruded and laminated, or co-extruded as melts and co-solidified to make a multi-layer film which can be formed into coverings such as bags, cylinders or tubes. This arrangement enables a gas (e.g., chlorine dioxide) atmosphere to be provided over a period of days, weeks or months. Suitable water-insoluble, water-permeable films can be composed of poly(ethylene-propylene) or poly(acrylic-ester acrylate) copolymers or monomers thereof such as sulfonated salts of poly(ethylene-propylene). Hydroxyethylmethacrylate, methoxyethylmethacrylate polymers and copolymers and other polymers form water-insoluble, water-permeable films well known in the art that are also suitable.

In another embodiment, the compositions of the present invention can be used in forming a multilayered composite, such as a film, wherein the compatible polymer blend of the invention forms an exposed layer and one or more non-active layers are co-extruded with the active layer. The non-active layer or layers may impart mechanical strength to the composite that could not be achieved by the compatible polymer blend layer. Composites of the invention may be separately extruded and laminated, or co-extruded as melts and co-solidified to make a multi-layer film. The composite can then be formed into a covering such as a tube, bag or wrapping wherein the active layer is the inner layer and is directly exposed to the contents of the covering.

In another embodiment, the first and/or third layers may contain an acid releasing compound while the second layer contains the anions (i.e., the anions and the acid releasing hydrophobic polymer or acid releasing agent are not admixed). Generally, any acid releasing polymer, or polymer that contains an acid releasing agent, that can be melt extruded at temperatures compatible with the composite of the invention to give a transparent or translucent layer having the required mechanical and theological properties may be used.

The layered composites of the present invention are intended to maintain a desired rate of gas release (moles/sec/cm² of film) in the presence of atmospheric moisture at a surface for a length of time required for the gas to absorb onto the surface and kill bacteria or other microbiological contaminants. The gas concentration released from the film for a chosen time period can be calculated given the release rate. Thus after measuring the release rate, the composite is formulated so that it contains a large enough reservoir of gas-generating anions reacting at a rate sufficient for the desired time period of sustained release.

Applications for the compositions of the invention are numerous. The compositions can be used in most any environment where exposure to moisture with subsequent release of gas such as chlorine dioxide can occur. The compositions can be melt processed into films, fibers, laminated coatings, tablets, tubing, pellets, powders, membranes, engineered materials, adhesives and multi film tie layers for a wide range of end uses. The compositions are particularly useful in preparing injection, compression, thermoform, extrusion or blow molded products. The melt can be applied on a surface as a film by using hot melt dip or lamination processes known in the art.

The water-activated compositions can be used in most any environment where exposure to moisture will occur. The compositions can be used to prevent the growth of molds, fungi, viruses and bacteria on the surface of a material, deodorize the material or inhibit infestation by treating a surface of a substrate with a composition that does not release a gas in the absence of moisture, and exposing the treated surface to moisture to release the gas from the composition into the atmosphere surrounding the surface. The release of the gas retards bacterial, fungal, and viral contamination and growth of molds on the surface, deodorizes the surface, and inhibits infestation.

The compositions of the present invention are particularly useful for the manufacture of devices, containers or film wraps. For example, formed containers or films may be used to generate a biocidal atmosphere for storing and displaying food products including blueberries, raspberries, strawberries, and other produce, ground beef patties, chicken filets, and other meats, enhanced foods, pet foods, dry foods, cereals, grains, or most any food subject to bacterial contamination or mold growth, algae or fungus. Additionally, soap, laundry detergents, documents, clothing, paint, seeds, medical instruments, food-ware, personal care products, biological or medical waste, refuse, or other medical, home and commercial products, may also be stored and sterilized by compositions of the invention. Devices such as catheters, sutures, tracheotomy tubes, syringes, or generally any polymer-based medical device or product may be manufactured with the composition of the invention. Moreover, bandage material, body covering articles such as gloves or garments, shower curtains, or generally any application requiring a film composition can be produced with the composition. The compositions are especially useful for applications requiring maximum transparency, such as surgical bandages permitting the observation of healing, or food wraps that permit the observation of food quality. Further applications include forming extruded chlorine dioxide releasing rods for use as a decontamination additive for water or water based drink products. Foamed composition products can the used as packaging material that generates a biocidal atmosphere and protects against mechanical shock.

Surfaces can be treated with a composition of the present invention by conventional coating, extrusion, lamination and impregnation methods well known in the art. The treated surface is generally a portion of a container, a part of a substrate placed within a container, or a packaging film or other type of packaging. When an optically transparent composition of the invention has been applied to a substrate, the substrate surface can clearly be seen through the film formed on the surface. If the composition, for example, is coated onto a containerboard box printed with graphics, the graphics remain clearly visible. A container or substrate can be protected with a coating of the biocidal composition although the composition is transparent and virtually unnoticeable to a consumer.

### DEFINITIONS

For purposes of the present invention, the term “compatible polymer blend” means a polymer blend where there is a sufficient interphase mixing and favorable interaction between the components so that the blend exhibits at least macroscopically uniform physical properties throughout its whole volume.

The term “hydrocarbon” as used herein describes organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbon” moieties described herein are hydrocarbon moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, aryloxy, hydroxy, protected hydroxy, acyl, acyloxy, nitro, amino, amido, nitro, cyano, ketals, acetals, esters and ethers.

Where the term “alkyl” is used, either alone or with another term such as “haloalkyl” and “alkylsulfonyl”, it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of such radicals include ethenyl, -propenyl, butenyl, and the like.

The terms “alkanoyl” or “carboxyalkyl” embrace radicals having a carboxy radical as defined above, attached to an alkyl radical. The alkanoyl radicals may be substituted or unsubstituted, such as formyl, acetyl, propionyl (propanoyl), butanoyl (butyryl), isobutanoyl (isobutyryl), valeryl (pentanoyl), isovaleryl, pivaloyl, hexanoyl or the like.

The term “alkoxy” embraces linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The “alkoxy” radicals may be further substituted with one or more halogen atoms, such as fluoro, chloro or bromo, to provide “haloalkoxy” radicals. Examples of such radicals include fluoromethoxy, chloromethoxy, trifluoromethoxy, trifluoroethoxy, fluoroethoxy and fluoropropoxy.

The term “alkoxycarbonyl” means a radical containing an alkoxy radical, as defined above, attached via an oxygen atom to a carbonyl radical. Preferably, “lower alkoxycarbonyl” embraces alkoxy radicals having one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl.

The term “alkylaminoalkyl” embraces aminoalkyl radicals having the nitrogen atom substituted with an alkyl radical.

The term “alkylcarbonyl” embraces radicals having a carbonyl radical substituted with an alkyl radical. More preferred alkylcarbonyl radicals are “lower alkylcarbonyl” radicals having one to six carbon atoms. Examples of such radicals include methylcarbonyl and ethylcarbonyl.

The term “alkylcarbonylalkyl”, denotes an alkyl radical substituted with an “alkylcarbonyl” radical.

The term “aminocarbonyl” denotes an amide group of the formula —C(═O)NH₂.

The term “aminoalkyl” embraces alkyl radicals substituted with amino radicals.

The term “aralkyl” embraces aryl-substituted alkyl radicals. Preferable aralkyl radicals are “lower aralkyl” radicals having aryl radicals attached to alkyl radicals having one to six carbon atoms. Examples of such radicals include benzyl, diphenylmethyl, triphenylmethyl, phenylethyl and diphenylethyl. The aryl in the aralkyl may be additionally substituted with halo, alkyl, alkoxy, halkoalkyl and haloalkoxy.

The term “sulfinyl” embraces a divalent —S(═O)— moiety.

The term “aryl”, alone or in combination, means a carbocyclic aromatic system containing one, two or three rings wherein such rings may be attached together in a pendent manner or may be fused. The term aryl embraces aromatic radicals such as phenyl, naphthyl, tetrahydronaphthyl, indane and biphenyl.

The term “arylamino” denotes amino groups which have been substituted with one or two aryl radicals, such as N-phenylamino. The arylamino radicals may be further substituted on the aryl ring portion of the radical.

The term “acyl”, whether used alone, or within a term such as “acylamino”, denotes a radical provided by the residue after removal of hydroxyl from an organic acid.

The terms “carboxy” or “carboxyl”, whether used alone or with other terms such as “carboxyalkyl”, denotes —CO₂H.

The term “carbonyl”, whether used alone or with other terms, such as “alkylcarbonyl”, denotes —(C═O)—.

The term “cycloalkenyl” embraces unsaturated cyclic radicals having three to ten carbon atoms, such as cyclobutenyl, cyclopentenyl, cyclohexenyl and cycloheptenyl.

The term “cycloalkyl” embraces radicals having three to ten carbon atoms. More preferred cycloalkyl radicals are “lower cycloalkyl” radicals having three to seven carbon atoms. Examples include radicals such as cyclopropyl, cyclobutyl, cyclopentyl, cyclohexyl and cycloheptyl.

The term “ester” includes alkylated carboxylic acids or their equivalents, such as (RCO-imidazole).

The term “halo” means halogens such as fluorine, chlorine, bromine or iodine atoms.

The term “heteroaryl” embraces unsaturated heterocyclic radicals including unsaturated 3 to 6 membered heteromonocyclic groups containing nitrogen, oxygen or sulfur atoms. The term also embraces radicals where heterocyclic radicals are fused with aryl radicals. Examples of such fused bicyclic radicals include benzofuran, benzothiophene, and the like.

The term “heterocyclo” embraces saturated, partially saturated and unsaturated heteroatom-containing ring-shaped radicals, where the heteroatoms may be selected from nitrogen, sulfur and oxygen.

The term “hydration” refers to the uptake of water. The term “hydrolysis” refers to the reaction of water with another substance to form two or more new substances, for example the reaction of an acid releasing substance or moiety with water to form hydronium ion, H₃O⁺.

The term “hydronium” or “hydronium ion” is H₃O⁺.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “sulfamyl,” “aminosulfonyl” and “sulfonamidyl”, denote a sulfonyl radical substituted with an amine radical, forming a sulfonamide substituted with an amine radical, forming a sulfonamide (—SO₂NH₂).

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals include methylsulfonyl, ethylsulfonyl and propylsulfonyl. The term “arylsulfonyl” embraces aryl radicals as defined above, attached to a sulfonyl radical. Examples of such radicals include phenylsulfonyl.

The following examples are presented to describe preferred embodiments and utilities of the present invention and are not meant to limit the present invention unless otherwise stated in the claims appended hereto.

### EXAMPLE 1

The ClO₂ releasing properties of co-extruded three and two layer films were evaluated. The films incorporated a moisture activated ClO₂ active layer and two barrier layers. In a first embodiment, the active layer was co-extruded between barrier layers (i.e., the active layer was the middle layer). In a second embodiment, the active layer was co-extruded as an exposed layer intended to form the inner layer of a tube or bag (i.e., the active layer was the inner layer). The outer layers consisted of the same material (Lupolen® 1806H). The components used for the trials are described in Table 1. Typical extrusion parameters are shown in Table 2 which describes the parameters used to prepare trial film number 006 wherein each layer was extruded on a separate extruder. In general, the active layer was prepared by melt extruding a dry blend of one or more polymers, sodium chlorite and a plasticizer. The composition of all of the films evaluated in this example is described in Table 3.

TABLE 1

| Component | Description |
|---|---|
| Lupolen ® 1806 H | Standard low density polyethylene (density of 0.92 g/cm³); |
| (available from | melt flow index (MFI) = 1.6; melting point = 109° C.; |
| Basell GmBH) | containing slip (erucamide) and anti-block |
| | (natural silica) additives. |
| Surlyn ® 1652 (available | Ionomer (Zn); MFI = 5; melting point = 100° C. |
| from DuPont ®) | |
| Active Resin 1 | Dry blend of 20 wt % PEOX (Aquazol-50); 2 wt % DBP |
| | (dibutyl phthalate); 3 wt % (sodium chlorite); 50 wt % |
| | PEAA (Nucrel 2806); and 25 wt % ethyelene vinyl |
| | alcohol (EVA)(Elvax 3170). |
| Active Resin 2 | Dry blend of 30 wt % PEOX (Aquazol-50); 2 wt % DBP |
| | (dibutyl phthalate); 3 wt % (sodium chlorite); 50 wt % |
| | PEAA (Nucrel 2806); and 15 wt % EVA (Elvax 3170). |
| Active Resin 3 | Dry blend of 30 wt % PEOX (Aquazol-50); 2 wt % DBP |
| | (dibutyl phthalate); 5 wt % (sodium chlorite); 50 wt % |
| | PEAA (Nucrel 2806); and 13 wt % EVA (Elvax 3170). |

TABLE 2

| | Extruder 2 | Extruder 1 | Extruder 3 | |
|---|---|---|---|---|
| | Outer Layer 1 | Middle Layer | Outer Layer 2 | |
| | Lupolen | Active resin 2 | Lupolen | |
| Material | 1806H | | 1806H | Die Head |
| Extruder Temperature Setting by Zone | | | | ° C. |
| 1 | 125° C. | 90° C. | 100° C. | 130° C. |
| 2 | 130° C. | 95° C. | 130° C. | 130° C. |
| 3 | 130° C. | 95° C. | 130° C. | 130° C. |
| 4 | 130° C. | 95° C. | 130° C. | 130° C. |
| 5 | 130° C. | 95° C. | 130° C. | 130° C. |
| 6 | 130° C. | 95° C. | 130° C. | 130° C. |
| 7 | 130° C. | 95° C. | 130° C. | 130° C. |
| 8 | 130° C. | 100° C. | — | 130° C. |
| Rotation speed | 80 rev/min | 16 rev/min | 35 rev/min | — |
| Melt Temperature | 146° C. | 102° C. | 128° C. | — |
| Current Consumption | 12.4 amps | 3 amps | 0.7 amps | — |
| Melt Pressure | 267 bar | 141 bar | 205 bar | — |

TABLE 3

| | Outer Layer 1 | | Middle Layer | | Outer Layer 2 | | Total |
|---|---|---|---|---|---|---|---|
| Trial No. | material | thickness | material | thickness | material | thickness | thickness |
| 001 | Lupolen | 10 μm | Lupolen | 15 μm | Active | 25 μm | 50 μm |
| | 1806H | | 1806H | | resin 2 | | |
| 002 | Lupolen | 10 μm | Lupolen | 20 μm | Active | 10 μm | 40 μm |
| | 1806H | | 1806H | | resin 2 | | |
| 003 | Lupolen | 10 μm | Lupolen | 20 μm | Active | 10 μm | 40 μm |
| | 1806H | | 1806H | | resin 1 | | |
| 004 | Lupolen | 10 μm | Lupolen | 20 μm | Active | 10 μm | 40 μm |
| | 1806H | | 1806H | | resin 3 | | |
| 005 | Lupolen | 10 μm | Lupolen | 15 μm | Active | 25 μm | 50 μm |
| | 1806H | | 1806H | | resin 3 | | |
| 006 | Lupolen | 17 μm | Active | 16 μm | Lupolen | 7 μm | 40 μm |
| | 1806H | | resin 2 | | 1806H | | |
| 007 | Lupolen | 17 μm | Active | 16 μm | Surlyn | 7 μm | 40 μm |
| | 1806H | | resin 2 | | 1652 | | |
| 008 | Lupolen | 17 μm | Active | 16 μm | Surlyn | 7 μm | 40 μm |
| | 1806H | | resin 1 | | 1652 | | |
| 009 | Lupolen | 17 μm | Active | 16 μm | Surlyn | 7 μm | 40 μm |
| | 1806H | | resin 3 | | 1652 | | |
| 010 | Lupolen | 25 μm | Active | 5 μm | Surlyn | 5 μm | 35 μm |
| | 1806H | | resin 2 | | 1652 | | |
| 011 | Lupolen | 18 μm | Lupolen | 12 μm | Active | 20 μm | 40 μm |
| | 1806H | | 1806H | | resin 2^{a} | | |

^{a}98 wt % Active resin 2 and 2 wt % talc

Force and elongation at break of trial numbers 002, 006, 007 and 010 were measured using a standard tensile tester (Zwick® 1425) with 50 mm×15 mm sample sizes. Speed of elongation was fixed at 500 mm/min. The results are reported in Table 4 where MD is machine direction and TD is transverse direction.

TABLE 4

| | | | | Elongation | | Tensile | |
|---|---|---|---|---|---|---|---|
| | | Force at | | at | | Strength | |
| | | Break (N) | | Break (N) | | (N/mm²) | |
| Trial No. | Thickness | MD | TD | MD | TD | MD | TD |
| 002 | 38 μm | 15.0 | 13.0 | 170 | 370 | 26.2 | 22.8 |
| 006 | 38 μm | 11.6 | 8.0 | 170 | 300 | 20.3 | 14.0 |
| 007 | 40 μm | 14.1 | 10.1 | 170 | 305 | 23.5 | 16.8 |
| 010 | 37 μm | 14.9 | 11.4 | 165 | 360 | 26.8 | 20.4 |
| Lupolen 1806H^{a} | 50 μm | 20.3 | 12.8 | 200 | 600 | 27.0 | 17.0 |

^{a}Nominal values for 50 μm blown mono film as given by Basell from the product data sheet.

The mechanical properties should be sufficient for the preparation of a standard waste bag having a volume of op to 35 liters.

The ClO₂ releasing properties of the films described in Table 3 at various levels of humidity is reported in Tables 5-10. ClO₂ levels were measured using electrochemical (EC) gas sensors (Citicel 3MCLH), each of which was calibrated to measure in the part per million (ppm or μL/L) range. Concentration data from 8 sensors was continuously recorded by computer over the indicated periods of time using a data acquisition module (Iotech) and control software (Labview). An EC sensor was mounted in the lid of a 250 ml glass jar containing a small plastic cup holding a suitable constant humidity source. For example, a saturated ammonium sulfate solution was used to generate a relative humidity of about 80% inside the jar.

Rectangular film samples weighing about 1 gram and measuring about 18 cm×12-14 cm were cut from the larger co-extruded film. The film sample was placed in the jar and the lid/sensor assembly was then secured to the jar which was then placed in an enclosure thermostatically controlled at 21° C. The data acquisition system was then activated and ClO₂ concentration was measured and recorded every 5 minutes thereafter. The results are reported in Tables 5-10 below.

TABLE 5
ClO₂ release of three layer films evaluated at 80% RH

| | Trial 006 | Trial 008 | Trial 009 | Trial 010 |
|---|---|---|---|---|
| Days | (ppm ClO₂)^{a} | (ppm ClO₂)^{b} | (ppm ClO₂)^{c} | (ppm ClO₂)^{d} |
| 0.5 | 0.1 | 2.0 | 0.5 | 0.8 |
| 1 | 1.1 | 8.7 | 2.3 | 2.1 |
| 1.5 | 4.7 | 10.5 | 6.6 | 2.6 |
| 2 | 8.7 | 9.0 | 11.0 | 2.8 |
| 2.5 | 10.8 | 7.2 | 13.0 | 2.6 |
| 3 | 11.5 | 5.9 | 13.8 | 2.5 |
| 3.5 | 10.3 | 4.8 | 12.7 | 2.3 |
| 4 | 9.1 | 4.1 | 12.1 | 2.1 |
| 4.5 | 7.5 | 3.3 | 10.5 | 1.8 |
| 5 | 6.2 | 2.8 | 9.3 | 1.7 |
| 5.5 | 5.1 | 2.3 | 8.2 | 1.5 |
| 6 | 4.1 | 2.0 | 7.4 | 1.3 |
| 6.5 | 3.2 | 1.7 | 6.4 | 1.2 |
| 7 | 2.5 | 1.5 | 6.0 | 1.1 |
| 7.5 | 1.9 | 1.3 | 5.1 | 1.0 |
| 8 | 1.7 | 1.1 | 4.8 | 1.0 |
| 8.5 | 1.3 | 1.0 | 4.0 | 0.8 |
| 9 | 1.1 | 0.9 | 3.8 | 0.7 |
| 9.5 | 0.9 | 0.8 | 3.1 | 0.6 |

^{a}maximum release: 11.5 ppm at 3 days.
^{b}maximum release: 10.7 ppm at 1.4 days.
^{c}maximum release: 13.8 ppm at 3 days.
^{d}maximum release: 2.8 ppm at 2 days.

TABLE 6
ClO₂ release of trial 007 three layer film evaluated at 0%, 60%, 80% and
100% RH

| | 0% RH | 60% RH | 80% RH | 100% RH |
|---|---|---|---|---|
| Days | (ppm ClO₂) | (ppm ClO₂) | (ppm ClO₂)^{a} | (ppm ClO₂)^{b} |
| 0.5 | 0.0 | 0.0 | 0.8 | 9.5 |
| 1 | 0.0 | 0.1 | 9.9 | 14.7 |
| 1.5 | 0.0 | 0.2 | 21.5 | 6.2 |
| 2 | 0.1 | 0.3 | 14.8 | 3.7 |
| 2.5 | 0.2 | 0.6 | 8.4 | 2.3 |
| 3 | 0.3 | 0.8 | 5.2 | 1.8 |
| 3.5 | 0.5 | 0.9 | 3.5 | 1.4 |
| 4 | 0.6 | 1.0 | 2.6 | 1.2 |
| 4.5 | 0.6 | 1.1 | 2.0 | 1.0 |
| 5 | 0.6 | 1.1 | 1.6 | 0.8 |

^{a}maximum release: 21.5 ppm at 1.5 days.
^{b}maximum release: 18.6 ppm at 0.7 days.

TABLE 7
ClO₂ release of trial 001 film evaluated at 0%, 60%, 80% and 100% RH

| | 0% RH | 60% RH | 80% RH | 100% RH |
|---|---|---|---|---|
| Days | (ppm ClO₂) | (ppm ClO₂) | (ppm ClO₂)^{a} | (ppm ClO₂)^{b} |
| 0.5 | 0.1 | 0.2 | 0.8 | 0.2 |
| 1 | 0.1 | 0.3 | 6.8 | 54.5 |
| 1.5 | 0.2 | 0.4 | 15.2 | 16.1 |
| 2 | 0.4 | 0.4 | 18.4 | 8.1 |
| 2.5 | 0.6 | 0.5 | 16.8 | 5.2 |
| 3 | 0.8 | 0.6 | 14.9 | 3.8 |
| 3.5 | 1.0 | 0.6 | 12.1 | 2.9 |
| 4 | 1.2 | 0.7 | 10.2 | 2.4 |
| 4.5 | 1.3 | 0.8 | 8.1 | 2.1 |
| 5 | — | — | 6.6 | 1.8 |
| 5.5 | — | — | 5.2 | — |
| 6 | — | — | 4.3 | — |
| 6.5 | — | — | 3.4 | — |
| 7.0 | — | — | 2.9 | — |
| 7.5 | — | — | 2.3 | — |
| 8.0 | — | — | 2.0 | — |
| 8.5 | — | — | 1.5 | — |
| 9 | — | — | 1.4 | — |

^{a}maximum release: 18.4 ppm at 2 days.
^{b}maximum release: 66.3 ppm at 0.4 days.

TABLE 8
ClO₂ release of two layer films evaluated at 80% RH

| | Trial 002 | Trial 003 | Trial 011 |
|---|---|---|---|
| Days | (ppm ClO₂)^{a} | (ppm ClO₂)^{b} | (ppm ClO₂)^{c} |
| 0.5 | 2.8 | 0.3 | 1.8 |
| 1 | 12.0 | 0.8 | 5.3 |
| 1.5 | 19.0 | 1.3 | 8.4 |
| 2 | 17.6 | 1.6 | 7.6 |
| 2.5 | 13.7 | 1.8 | 5.5 |
| 3 | 10.9 | 1.9 | 3.9 |
| 3.5 | 7.6 | 1.9 | 2.9 |
| 4 | 5.0 | 2.1 | 2.4 |
| 4.5 | 3.1 | 2.0 | 1.8 |
| 5 | 2.2 | 2.0 | 1.5 |
| 5.5 | 1.7 | 2.0 | 1.2 |
| 6 | 1.3 | 2.0 | 1.1 |
| 6.5 | 1.1 | 1.9 | 0.9 |
| 7 | 1.0 | 2.0 | 0.8 |
| 7.5 | 0.8 | 1.8 | 0.7 |
| 8 | 0.7 | 1.9 | 0.7 |
| 8.5 | 0.6 | 1.7 | 0.6 |
| 9 | 0.6 | 1.8 | 0.5 |
| 9.5 | 0.4 | 1.5 | 0.4 |

^{a}maximum release: 19.1 ppm at 1.6 days.
^{b}maximum release: 2.1 ppm at 4 days.
^{c}maximum release: 8.6 ppm at 1.7 days.

TABLE 9
ClO₂ release of two layer film 005 evaluated at various RH

| | Trial 005 | | |
|---|---|---|---|
| | (ppm ClO₂ at | Trial 005 (ppm ClO₂ | Trial 005 (ppm ClO₂ |
| Days | 0% RH) | at 60% RH) | at 80% RH)^{a} |
| 0.5 | 0.0 | 0.5 | 1.1 |
| 1 | 0.1 | 0.5 | 6.3 |
| 1.5 | 0.1 | 0.7 | 19.6 |
| 2 | 0.2 | 0.8 | 34.3 |
| 2.5 | 0.3 | 0.9 | 41.7 |
| 3 | 0.5 | 1.0 | 45.2 |
| 3.5 | 0.7 | 1.1 | 42.0 |
| 4 | 1.0 | 1.2 | 38.3 |
| 4.5 | 1.0 | 1.3 | 31.1 |
| 5 | 0.5 | 1.4 | 23.7 |
| 5.5 | 0.2 | 1.3 | 17.6 |
| 6 | 0.0 | 1.4 | 13.9 |
| 6.5 | 0.0 | 1.4 | 10.8 |
| 7 | 0.0 | 1.4 | 9.1 |
| 7.5 | 0.0 | 1.4 | 7.3 |
| 8 | 0.0 | 1.4 | 6.4 |
| 8.5 | 0.0 | 1.3 | 5.1 |
| 9 | 0.0 | 1.3 | 4.5 |
| 9.5 | 0.0 | 1.3 | 3.6 |

^{a}maximum release: 47.0 ppm at 3.04 days.

TABLE 10
ClO₂ release of two layer film 004 at 0% and 80% RH

| | Trial 004 (ppm | Trial 004 |
|---|---|---|
| Days | ClO₂) at 0% RH | (ppm ClO₂) at 80% RH^{a} |
| 0.5 | 0.0 | 0.9 |
| 1 | 0.1 | 5.1 |
| 1.5 | 0.2 | 11.3 |
| 2 | 0.4 | 15.2 |
| 2.5 | 0.6 | 10.2 |
| 3 | 0.6 | 5.6 |
| 3.5 | 0.6 | 3.3 |
| 4 | 0.6 | 2.3 |
| 4.5 | 0.5 | 1.5 |
| 5 | — | 1.2 |
| 5.5 | — | 0.9 |
| 6 | — | 0.8 |
| 6.5 | — | 0.7 |
| 7 | — | 0.6 |
| 7.5 | — | 0.5 |
| 8 | — | 0.5 |
| 8.5 | — | 0.4 |
| 9 | — | 0.4 |
| 9.5 | — | 0.3 |

^{a}maximum release: 15.2 ppm at 2 days.

### EXAMPLE 2

### Visula Inspection and Optical Microscopy of PEAA-PEOX Hydrophilic Polymer Blends

To test the compatibility of PEOX and the ethylene-acrylic and ethylene-methacrylic copolymers PEAA 15, PEAA 20 (Dow Primacor low molecular weight ethylene acrylic acid copolymer (20 wt % acrylic acid co-monomer)) and poly (ran-ethylene-methacrylic acid) (PEMAA) respectively, separate THF solutions of 5,000 and 50,000 dalton MW PEOX (available from Polymer Chemistry Innovations) and the copolymers (available from Aldrich) were mixed in the correct proportions to make a casting solution. Although PEOX dissolved rapidly in THF at room temperature, PEAA 15 and PEAA 20 were relatively insoluble at room temperature in THF and required boiling the THF for complete dissolution.

Films were initially made by casting from THF solution on glass slides. As shown in Table 11, the films that were dried quickly in warm air were optically transparent while slowly dried film exhibited some translucency which could be removed by heating to 80° C. for 12 hrs under vacuum. This temperature is above the T_{g} of either component and at the Tₘ of the ethylene component of the acrylate copolymer.

In addition to being transparent, the films containing at least 50 wt % of the copolymer were tough and rubbery and could be stretched several hundred percent prior to fracture. Unplasticized, unblended PEOX was brittle at room temperature.

TABLE 11
Optical observations on PEOX-PEAA (PEMAA) blend compatibility after
annealing for 12 hrs at 80° C.

| | % PEOX | PEAA 15 | PEAA 20 | PEMAA 15 |
|---|---|---|---|---|
| PEOX 5 | 50% | — | — | Clear |
| PEOX 50 | 30% | Clear | Clear | — |
| PEOX 50 | 40% | Clear | Clear | — |
| PEOX 50 | 50% | Clear | Clear | — |
| PEOX 50 | 70% | Clear | Clear | — |

In table 11, PEOX 5 and PEOX 50 are poly(ethyloxazoline) of 5,000 MW and poly(ethyloxazoline) of 50,000 MW, respectively. PEAA 15 and PEAA 20 are poly (ran-ethylene-acrylic acid) containing 15 wt % acrylic acid and 85 wt. % ethylene and poly (ran-ethylene-acrylic acid) containing 20 wt % acrylic acid and 80 wt. % ethylene, respectively. PEMAA 15 is poly (ran-ethylene-methacrylic acid) containing 15 wt % methacrylic acid and 85 wt. % ethylene, respectively.

### EXAMPLE 3

### Swelling of Compression Molded PEOX-PEAA Film with Water

A strip of the compression molded 60% PEAA20-40% PEOX 50 film weighing 0.3690 grams and an average thickness of 0.3 mm was immersed in de-ionized water for one hour at room temperature. The film increased in weight by 35% to 0.4985 grams and increased in thickness by 8.3% to 0.325 mm and remained elastomeric. The water-swelled film was basically transparent with a slight cloudiness suggesting an IPN morphology.

### EXAMPLE 4

### PEOX Stability with Acidic Chlorine Dioxide Solutions

Acidic water solutions of sodium chlorite and PEOX 50 were monitored over several hours at 25° C. by UV-Visible spectrometry. No degradation of the PEOX 50 was observed during this time. In addition, a sample of chlorine dioxide in a water solution of excess PEOX showed no color change over two weeks at 25° C. indicating little if any reaction of the chlorine dioxide with PEOX.

### EXAMPLE 5

### PEOX Stability in Basic Solution

Water solutions of Textone (i.e., sodium chlorite) are typically basic and the long term stability of chlorite anions in solution is believed to be dependent upon a basic pH. It is further believed that even low concentrations of protons can result in the formation of small amounts of chlorous acid which is unstable and disproportionation to chlorine dioxide is favored.

Some chlorite decomposition was observed in blends of PEOX and Textone that were cast from water. Under one theory, and without being bound to any particular theory, and based upon observations to date, it is believed that chlorite can be complexed by the PEOX amide groups as water is evaporated promoting reaction with protons to form chlorous acid. It is further believed that addition of a hydroxide anion to the mixture could stabilize the chlorite, but also could potentially cleave the amide portion of the PEOX. To evaluate that mechanism, a water solution of PEOX in sodium hydroxide (pH>11) was stirred overnight and analyzed by proton nuclear magnetic resonance (HNMR). The spectrum of the exposed material was essentially identical to that of a PEOX standard indicating stability of chlorite in basic solution with PEOX. HNMR additionally showed stability of PEOX in basic solution as no trace of the propionic acid that would have resulted from a cleavage of the amide portion of PEOX bond was found.

### EXAMPLE 6

### Preparation of Solvent Cast Blends of PEOX, NaOh and Textone or Sodium Polyphosphate

About 0.25 g/ml of PEOX 50 in methanol and about 0.33 g/ml total of combined NaOH and Textone were combined in various mixing ratios. The combined solution briefly turned cloudy before clearing. The solutions were immediately transferred into stainless steel pans to a depth of about 0.5″ and then vacuum dried for about 10 hours at a temperature of about 50° C. During the drying process the material foamed into a brittle transparent glass which was easily crushed into a fine powder.

Water solutions of PEOX 50 and NaOH, with added Textone, sodium polyphosphate (NaPO₃-Calgon) or sodium dihydrogen phosphate were prepared in a similar manner except that vacuum evaporation at 70° C. was employed. PEOX-polyphosphate glasses cast from water were transparent up to 15 wt % inorganic component. Table 12 tabulates the cast compositions.

TABLE 12
Compositions of Solvent Cast Blends of Inorganic Components
in PEOX 50

| Test No. | Description of Vacuum Cast Films |
|---|---|
| 1 | 8% Textone in PEOX 50K cast from H2O at 60° C. |
| 2 | 8% NaH₂PO₄ (SPMB) in PEOX 50K cast from H2O at 70° C. |
| 3 | 8% Textone in PEOX 50K cast from MeOH at 50° C. 0.087 |
| 4 | 8% sodium polyphosphate (SPP) in PEOX 50K cast |
| | from H2O, 50° C. |
| 5 | 8% Textone, 0.5% NaOH in PEOX 50K cast from |
| | MeOH at 50° C. |
| 6 | 23% SPP in PEOX 50K cast from H2O at 57° C. |
| 7 | 8% Textone, 3% NaOH in PEOX 50K cast from MeOH |
| | at 50° C. |
| 8 | 8% Textone, 6% NaOH in PEOX 50K cast from MeOH |
| | at 50° C. |

### EXAMPLE 7

### Chlorite Stability During Casting of PEOX 50, Textone and NaOH Blends From Water and Methanol

Transparent glasses containing PEOX 50, Textone and NaOH can be produced by vacuum evaporation of either water or methanol solutions overnight at 50° C. and 70° C., respectively. The percentage of remaining chlorite (Textone) in powders and extruded film was determined by conversion of iodine by the chlorite anion under acidic conditions, and then titration of the iodine back to iodide with a known concentration of sodium thiosulfate. Results are reported as a percentage of chlorite remaining.

Titration of the vacuum dried powders containing sodium hydroxide concentrations from 0 to 6 wt % immediately after cooling to room temperature showed that sodium chlorite survival during casting is dependent on sodium hydroxide concentration. Decomposition of chlorite anion was apparent in glasses containing less than 2 wt % sodium hydroxide that are cast from either methanol or water (Table 13). Water cast glasses had a yellow color and an odor of chlorine dioxide.

Glasses cast from methanol showed an increase in chlorite yield to about 87% after casting at 3 wt % NaOH with no improvement at higher base concentrations. A subsidiary maximum in the chlorite recovery obtained was apparent in water cast glasses around 2 wt % sodium hydroxide with the chlorite recovery gradually increasing at higher base concentrations.

TABLE 13
The Dependence of Chlorite Recovery on Sodium Hydroxide
Concentration in PEOX 50, Sodium Hydroxide, Textone Glasses.

| | | | |
|---|---|---|---|
| | mg ClO₂ | mg ClO₂ | recovery |
| Sample ID | (actual) | (theoretical) | actual (%)) |

Glasses Precipitated from Methanol

| | | | |
|---|---|---|---|
| Example 6 Test No. 3 | 4.01 | 7.82 | 51.3 |
| Example 6 Test No. 7 | 6.14 | 7.01 | 87.5 |
| Example 6 Test No. 8 | 5.88 | 7.25 | 81.1 |

Glasses Precipitated from Water (1)*

| | | | |
|---|---|---|---|
| 0% NaOH | 3.64 | 0.17 | 4.7 |
| 1% NaOH | 4.00 | 1.23 | 30.7 |
| 2% NaOH | 4.48 | 3.26 | 72.6 |
| 3% NaOH | 3.20 | 1.37 | 42.9 |
| 4% NaOH | 1.96 | 1.04 | 52.9 |
| 5% NaOH | 3.95 | 2.34 | 59.2 |
| 6% NaOH | 1.84 | 1.85 | 100.2 |

Glasses Precipitated from Water (2)*

| | | | |
|---|---|---|---|
| 0% NaOH | 20.93 | 0.26 | 1.3 |
| 1% NaOH | 22.63 | 11.81 | 52.2 |
| 2% NaOH | 23.68 | 12.34 | 52.1 |
| 3% NaOH | 24.78 | 11.09 | 44.8 |
| 4% NaOH | 6.72 | 3.44 | 51.2 |
| 5% NaOH | 20.46 | 9.06 | 44.3 |
| 6% NaOH Bottom | 7.44 | 22.97 | 308.6 |

*Water (1) and water (2) refer to two separate casting experiments.

### EXAMPLE 8

### Thermal Stability of Glasses Containing PEOX, Sodium Hydroxide and Textone

Glasses containing 89 wt % PEOX, 8 wt % Textone and 3 wt % NaOH were heated for 30 minutes at various temperatures to determine the thermal stability of the dispersed (dissolved) chlorite at elevated temperatures. The powders were then titrated according to the method of Example 7 to determine the remaining chlorite concentration (Table 14).

TABLE 14
Recovery of Chlorite From PEOX 50, NaOH (3 wt %) and Textone
(8 wt %) Glasses After Thermal Annealing

| | Oven | | mg ClO₂ | |
|---|---|---|---|---|
| Sample | Temp (C.) | mg ClO₂ (actual) | (theoretical) | actual % recovery |
| 1 | 30 | 14.6 | 15.9 | 91.8 |
| 2 | 150 | 12.0 | 13.6 | 87.9 |
| 3 | 165 | 8.5 | 10.1 | 84.1 |
| 4 | 170 | 5.4 | 7.5 | 72.7 |
| 5 | 180 | 3.9 | 11.0 | 35.4 |
| 6 | 200 | 1.0 | 4.8 | 21.1 |

### EXAMPLES 9-17

### Extrusion of PEOX-PEAA Blends

In Examples 9-17 a wide variety of extruded compatible polymer blends were prepared with (1) chlorite containing materials such as Textone® particulate (80%, sodium chlorite, 18% sodium chloride and 2% sodium carbonate), core (sodium polysilicate glass containing Textone®), Microsphere® (core material spray dried with alkali and alkaline earth polyphosphate), and finely dispersed blends thereof, (2) moisture activated, acid releasing compounds such as sodium polyphosphate (SPP), sodium dihydrogen phosphate (SPMB), alkenyl succinic anhydride (ASA), and (3) polyethylenes (Exceed PE and Exxon Mi 20) which served to improve mechanical properties.

The compatible polymer blend films were generally prepared by starting the extrusion with the PEAA (pellets) and PEOX (flakes) in the desired ratio and then subsequently adding the premixed inorganic components to the extruder hopper. Once the inorganic-organic mixture had entered the extruder, a final allotment of PEAA 20-PEOX 50 was added in the same ratio as that found in the initial loading in order to remove inorganic material from the extruder. This method was used to improve mixing where the extruder screws were precoated with polymer. However, even though the inorganic loaded material was introduced rapidly, some interdiffusion with the initial and final PEAA 20-PEOX 50 loaded was expected. Thus the concentration of inorganic material in the film rose, stabilized, and fell with extrusion time, but never reached the theoretical value.

Chlorine dioxide release from the formed polymer films was evaluated using a 0.5 gram to 1 gram sample of extruded film from what was expected to be the most active region of the extrudate. The measurement apparatus was as described in Example 1, however, in some cases there was significant chlorine dioxide leakage through the EC cells which varied from jar to jar depending on the quality of the seal formed by the combination of the jar lid, EC connection through the lid and the EC cell internal seals. All measurements were performed in a thermostatted oven at 28° C.

### EXAMPLE 9

### Neat Polymers and Their Blends

Transparent 10 mil films of 50/50, 60/40, and 70/30 (PEAA 20-PEOX 50) were produced using the twin screw extruder with a slotted mixing screw at 100° C. with a extrusion time of about 9 minutes. The high transparency was indicative of significant phase compatibility. These films were observed with an optical microscope under crossed polarizers and found to be quite birefringent. Without being bound to any particular theory, it is believed that birefringence is possibly indicative of a high degree of orientation in the machine direction, and the orientation may have been induced by the high take-up speed of the cooling rollers which were placed at the exit of the extruder.

PEOX 50 was easily extruded above 100° C. into a transparent film that was ductile at 37° C. but quite brittle below that temperature. The high torques required to drive the screws precluded effective extrusion of PEOX 50 at temperatures below 100° C. PEAA 20 and blend of PEAA with PEOX, on the other hand, could be extruded into a clear film at temperatures at 90° C. and higher temperatures.

### EXAMPLE 10

Microsphére® Blends

60/40 PEAA 20-PEOX 50 blends with 20 wt % of a chlorine dioxide releasing composition comprising a sodium polysilicate glass containing sodium chlorite (Microsphére® G71 and Prochem MS) were extruded at several temperatures (90° C.-120° C.). Optical microscope investigation revealed a profusion of ellipsoidal bubbles whose long axes were oriented in the extrusion direction in a highly birefringent film. The film readily fractured in the extrusion direction due to the stress concentrating effect of these bubbles, although this tendency was reduced upon exposure to moist air. Heating to 50° C. removed the birefringence and induced the bubbles to take spherical form. The origin of the bubbles is not precisely known, but it is believed, without being bound to any particular theory, that the bubbles were ClO₂ released by the Microsphére® during film preparation processing. Bubbles were seen even with extrusion temperatures of 90° C. and with extensively dried Microsphére®. There was also a tendency toward incomplete dispersal of the Microsphére®.

### EXAMPLE 11

### Core Blends

60/40 PEAA 20-PEOX 50 blends with 20 wt % of the core material of example 10 were extruded at 90° C. In the first case the PEAA served as the acid releasing agent. In a second case polyphosphate powder (dry ground in a food processor) was also added as an acid releasing agent during an extrusion at the same temperature. Cloudy brittle films were obtained.

### EXAMPLE 12

### Textone Powder (Coarse) Blends

60/40 PEAA 20-PEOX 50 blends with Textone (8 wt %-blender ground powder) and Textone (5 wt %) with the acid releasing compounds, sodium polyphosphate and sodium dihydrogen phosphate were extruded at 90° C. In all cases substantial number of elongated bubbles were seen which promulgated tearing along the machine direction.

### EXAMPLE 13

### PEOX Compatible Textone and Phosphate Blends

In this experiment, Textone (containing various amounts of sodium hydroxide) or phosphates that were solvent cast with PEOX 50 from either methanol or water were utilized as ground powders which were then mixed with appropriate amounts of PEAA prior to extrusion. In some cases from 20 wt % to about 70 wt % polyethylene (Exceed PE and Exxon MI 20 PE) was added to the mixture to improve film toughness. Finally from 3 wt % to 15 wt % alkenyl succinic anhydride (ASA) was added as an acid releasing agent, plasticizer and polyethylene compatibilizer to several blends.

Extruded films containing the predissolved Textone or polyphosphate were quite transparent and bubble free and had substantially better mechanical strength than the materials containing Textone particulate. The films containing polyethylene were translucent to transparent and demonstrated improved toughness; ASA further plasticized the films.

The high degree of optical transparency and improved toughness of these films suggests that the inorganic particles are smaller than 500 Å in diameter in the PEOX 50-PEAA blend.

TABLE 15
Concentration of Constituents in Extrudates-Absolute Component
Concentrations

| Test No. | Weight % Constituents |
|---|---|
| 1 | 20% Microsphere ® G71, 80% (60/40 PEAA-PEOX 50K) |
| 2 | 20% Prochem MS, 80% (60/40 PEAA-PEOX 50K) |
| 3 | 20% Prochem MS, 80% PEAA |
| 4 | 20% Prochem MS (vacuum dried), 80% PEAA |
| 5 | 20% Prochem MS (vacuum dried), 80% (60/40 PEAA-PEOX 50K) |
| 6 | 20% Prochem MS, 80% (60:40 PEAA-PEOX 5K) |
| 7 | 5% Textone (ground), 95% (60/40 PEAA-PEOX 50K) |
| 8 | 8% Textone (ground), 92% (60/40 PEAA-PEOX 50K) |
| 9 | 5% Textone (ground), 10.61% SPP, 84.39% (60/40 PEAA-PEOX 50K) |
| 10 | 5% Textone (ground), 10.61% SPMB, 84.39% (60/40 PEAA-PEOX 50K) |
| 11 | 3.36% Textone, 96.64% (60/40 PEAA-PEOX 50K) |
| 12 | 3.36% Textone, 96.64% (60/40 PEAA-PEOX 50K) |
| 13 | 3.36% SPP, 96.64% (60/40 PEAA-PEOX 50K) |
| 14 | 3.36% SPMB, 96.64% (60/40 PEAA-PEOX 50K) |
| 15 | 3.36% Textone, 0.21% NaOH, 96.43% (60/40 PEAA-PEOX 50K) |
| 16 | 10.67% SPP, 89.33% (60/40 PEAA-PEOX 50K) |
| 17 | 3.26% Textone, 3% NaOH, 93.74% (60/40 PEAA-PEOX 50K) |
| 18 | 3.47% Textone, 1.3% NaOH, 95.23% (59.4/40.6 PEAA-PEOX 50K) |
| 19 | 2.52% Textone, 3% ASA, 22% Exxon Mi 20 PE, 72.48% (60/40 PEAA- |
| | PEOX 50) |
| 20 | 1.68% Textone, 1.68% SPP, 96.4% (60/40 PEAA-PEOX 50K) |
| 21 | 349% Textone, 2.62% NaOH, 93.89% (60/40 PEAA-PEOX 50K) |
| 22 | 1.71% Textone, 1.28% NaOH, 1.71% SPP, 95.3% (60/40 PEAA-PEOX |
| | 50K) |
| 23 | 1.37% Textone, 1.03% NaOH, 1.37% SPP, 20% Mi 20 PE, 76.2% (60/40) |
| 24 | 3.42% Textone, 1.28% NaOH, 95.3% (60/40 PEAA-PEOX 50K) |
| 25 | 3.01% Textone, 1.13% NaOH, 12% ASA, 95.86% (60/40 PEAA-PEOX |
| | 50K) |
| 26 | 1.6% Textone, 0.6% NaOH, 10% ASA, 17.8% PEOX 50K, 70% Exceed PE |
| 27 | 3.2% Textone, 1.2% NaOH, 15% ASA, 35.6% PEOX 50K, 45% Exceed PE |
| 28 | 20% Microsphere ® core milled & dried, 80% (60/40 PEAA-PEOX 50K) |
| 29 | 20% Microsphere ® core milled & dried, 8.53% SPP, 71.47% (60/40 PEAA- |
| | PEOX 50K) |
| 30 | 8% Textone, 3% NaOH, 91% PEOX 50K plasticized with H2O |

TABLE 16
Extrusion Conditions and Film Morphology

| Test | | |
|---|---|---|
| No. | Temp (° C.) | Film Morphology |
| 1 | 100 | Transparent with undispersed solids, tears easily |
| 2 | 120 | Transparent with bubbles, tears easily |
| 3 | 120 | Bubbles, tears |
| 4 | 120 | Bubbles, brittle |
| 5 | 90 | Bubbles, tough but can tear and cleave |
| 6 | 90 | Light yellow colored film |
| 7 | 90 | Transparent with bubbles, tears easily |
| 8 | 90 | Transparent with bubbles, tears |
| 9 | 90 | Bubbles, undispersed solids, tears |
| 10 | 90 | Bubbles, undispersed solids, tears |
| 11 | 90 | Transparent, tough, tears longitudinally |
| 12 | 90 | Transparent with slight cloudiness, tough |
| 13 | 90 | Transparent |
| 14 | 90 | Clear with a few undispersed solids, tears |
| 15 | 90 | Transparent with some haziness, tough until tears |
| 16 | 90 | Undispersed solids, tough until tears |
| 17 | 90 | Transparent, tough until tears |
| 18 | 90 | Transparent with some unusual hazy patterns |
| 19 | 90 | Transparent and hazy, tough |
| 20 | 90 | Transparent and hazy, tough until tears |
| 21 | 90 | Transparent with some unusual hazy patterns |
| 22 | 90 | Transparent and hazy, appears unmixed, tough |
| | | until tears |
| 23 | 90 | Undispersed solids |
| 24 | 90 | Transparent and hazy, light bluish hue |
| 25 | 90 | Transparent with bubbles or solids, tough |
| | | until tears |
| 26 | 140 | Transparent with yellow hue |
| 27 | 140 | Transparent with yellow hue |
| 28 | 90 | Undispersed solids, tears |
| 29 | 90 | Undispersed solids |
| 30 | 0 | Clear and striated turned a brown color, |
| | | tears easily |

All extrusions were conducted on the conical twin screw extruder (screw repeat distance/screw length= 1/20) with a 20 rpm screw rate. The feed hopper was nitrogen gas purged for all extrusions.

### EXAMPLE 14

### Chlorite Content of Extruded Polymer Films

Example 13 test numbers 4 and 5, extruded films containing 20 wt % Microsphére® (Prochem MS-further vacuum dried at 100° C. 12 hrs) were extracted twice with dry peroxide free THF to remove polymeric components, and the inorganic powder was isolated. The titration procedure was used to determine that about 100% of the chlorite in the PEOX 50 based film (test number 5) survived the extrusion process at 120° C. while only 50% of the chlorite was present after extrusion of the PEAA film (test number 4) at the same temperature. This suggests that the carboxylic groups of the PEAA will react with the chlorite in Microsphére® to some extent at 120° C.

### EXAMPLE 15

Tables 17 and 18 represent 0.5 g samples of blend films containing Textone powder either without (Example 13, test no. 8) or with (Example 13, test no. 9) sodium polyphosphate tested at 80% RH and 58% RH respectively. These films contained much larger amounts of chlorite than the core containing film and thus showed release maxima more than 30× higher when tested at 80% RH. In all cases the maximum release took place around 20 hours at 80% RH. A small release maxima at 20 hours was followed by a larger broader maxima that appeared between 100-200 hours in materials tested at 58% RH. Although the experiment was stopped at 9 days, it is believed that the materials would have continued to generate chlorine dioxide for several weeks.

TABLE 17
Chlorine dioxide release at 80% RH

| Time (hrs) | Test No. 8 (ppm ClO₂) | Test No. 9 (ppm ClO₂) |
|---|---|---|
| 5 | 15 | 18 |
| 10 | 27 | 27 |
| 15 | 27 | 23 |
| 20 | 20 | 15 |
| 25 | 15 | 11 |
| 30 | 12 | 8 |
| 35 | 9 | 5 |
| 40 | 7 | 4 |
| 45 | 6 | 3 |
| 50 | 5 | 3 |
| 55 | 4 | 2.5 |
| 60 | 3 | 2 |
| 65 | 3 | 1.5 |
| 70 | 2.5 | 1.5 |
| 75 | 2 | 1 |
| 80 | 2 | 1 |
| 85 | 1.5 | 0.5 |
| 90 | 1.5 | 0.5 |

TABLE 18
Chlorine dioxide release at 58% RH

| Time (hrs) | Test No. 8 (ppm ClO₂) | Test No. 9 (ppm ClO₂) |
|---|---|---|
| 10 | 0.32 | 0.18 |
| 20 | 0.35 | 0.20 |
| 30 | 0.27 | 0.18 |
| 40 | 0.30 | 0.17 |
| 50 | 0.45 | 0.16 |
| 60 | 0.52 | 0.17 |
| 70 | 0.56 | 0.19 |
| 80 | 0.67 | 0.22 |
| 90 | 0.75 | 0.28 |
| 100 | 0.79 | 0.33 |
| 110 | 0.80 | 0.37 |
| 120 | 0.83 | 0.40 |
| 130 | 0.85 | 0.45 |
| 140 | 0.85 | 0.47 |
| 150 | 0.86 | 0.50 |
| 160 | 0.87 | 0.51 |
| 170 | 0.93 | 0.53 |
| 180 | 0.86 | 0.40 |
| 190 | 0.89 | 0.45 |
| 200 | 0.83 | 0.58 |

### EXAMPLE 16

In table 19 the effect of adding powdered phosphates to blends containing powered Textone was explored. A blend containing sodium polyphosphate (Example 13, test no. 9) appeared to be more active than the blends containing only Textone (Example 13, test no. 8) or sodium dihydrogen phosphate (Example 13, test no. 10), perhaps because of the hydroscopic nature of the polyphosphate. However, this comparison is only qualitative. Little or no activity was noticed for material containing predissolved Textone that was not stabilized by sodium hydroxide (Example 13, test no. 11).

TABLE 19
Chlorine dioxide release at 58% RH

| | Test No. 8 | Test No. 9 | Test No. 10 | Test No. 11 |
|---|---|---|---|---|
| Time (hrs) | (ppm ClO₂) | (ppm ClO₂) | (ppm ClO₂) | (ppm ClO₂) |
| 5 | 6.5 | 10.5 | 0.5 | 0 |
| 10 | 7.5 | 17.7 | 1.5 | 0 |
| 15 | 5.5 | 14.7 | 1.1 | 0 |
| 20 | 3.5 | 11.4 | 0.6 | 0 |
| 25 | 2.5 | 7.8 | 0.4 | 0 |
| 30 | 1.7 | 6.3 | 0.2 | 0 |
| 35 | 1.4 | 4.5 | 0 | 0 |
| 40 | 1.1 | 3.6 | 0 | 0 |
| 45 | 0.8 | 3.1 | 0 | 0 |
| 50 | 0.6 | 2.4 | 0 | 0 |
| 55 | 0.5 | 1.8 | 0 | 0 |
| 60 | 0.5 | 1.5 | 0 | 0 |
| 65 | 0.5 | 1.3 | 0 | 0 |
| 70 | 0.5 | 1.1 | 0 | 0 |
| 75 | 0.5 | 0.9 | 0 | 0 |
| 80 | 0.5 | 0.8 | 0 | 0 |
| 85 | 0.5 | 0.7 | 0 | 0 |
| 90 | 0.4 | 0.6 | 0 | 0 |
| 95 | 0.4 | 0.5 | 0 | 0 |
| 100 | 0.4 | 0.4 | 0 | 0 |

### EXAMPLE 17

Chlorine dioxide release of a transparent 60/40 PEAA-PEOX blend containing PEOX 50 and solubilized Textone stabilized by 3 wt % NaOH was tested at relative humidities of 58% and 80%. Example 13, test no. 18 demonstrated the best properties of any of the films (Table 20) in that substantial chlorine dioxide release (37 ppm) was observed from a reasonably tough transparent film. At 80% RH a large emission peak was observed followed by a long tail lasting several days. At 58% RH the emission level increased much more gradually over four days until 1 ppm was reached. The level of emission maintained this constant value for two weeks whereupon the emission rapidly decreased to zero.

TABLE 20

| | Test no. 18 (ppm ClO₂ | Test no. 18 (ppm ClO₂ |
|---|---|---|
| Time (days) | @80% RH) | @58% RH) |
| 0.5 | 38 | 0 |
| 1 | 9 | 0.3 |
| 2 | 4 | 0.5 |
| 3 | 2.2 | 0.7 |
| 4 | 1 | 1 |
| 5 | 0.3 | 1 |
| 6 | 0.1 | 1 |
| 7 | 0 | 1 |
| 8 | 0 | 1 |
| 9 | 0 | 1 |
| 10 | 0 | 1 |
| 11 | 0 | 1 |
| 12 | 0 | 1 |
| 13 | 0 | 1 |
| 14 | 0 | 1 |
| 15 | 0 | 1 |
| 16 | 0 | 1 |
| 17 | 0 | 0.5 |
| 18 | 0 | 0 |

### EXAMPLE 18

A composition of the present invention was evaluated in a commercial scale pelletizing operation.

A thoroughly mixed master batch was prepared by (i) admixing Aquazol-50 ethyl oxazoline hydrophilic polymer (PEOX) and dibutyl phthalate (DBP), (ii) admixing sodium chlorite powder having a nominal particle size of 20 microns with the PEOX-DBP mixture, and (iii) admixing Sasol Enhance 1585 wax having a molecular weight of about 1000 daltons (available from Sasol Wax (South Africa)) and DuPont Elvax 3170 hydrophilic ethylene vinyl alcohol hydrophobic polymer (EVA) with the PEOX-DBP-sodium chlorite mixture. The finished master batch contained about 40 wt % PEOX, about 6 wt % powdered sodium chlorite, about 4 wt % DBP, about 45% EVA and about 5 wt % wax. The master batch was added to a nitrogen blanketed feed hopper and extruded with a twin screw 30 mm extruder (Coperion Werner Pfleiderer GmbH & Co. model ZSK-30 compounder). Temperature was measured at four zones between the feed hopper and the extruder die, and at the extruder die. The temperature at the zone closest to the feed hopper (zone 1) was about 82-88° C., about 82-88° C. at zone 2, about 82-88° C. at zone 3, about 71-82° C. at zone 4 and about 107-121° C. at the extruder die. During extrusion the wax was observed to bloom at the extruded polymer surface.

From the die, the extruded master batch was immersed and cooled in a water bath to a temperature of less than about 27° C. at a residence time of about 3 to 5 seconds. The wax coating provided a barrier between the moisture activated polymer and the cooling water. Following cooling, excess water was removed from the surface of the extruded strand with an air knife. The extruded master batch was then cut into sections with a pelletizer. 395 kg of pelletized material was produced with about 90% chlorite recovery.

Films were prepared from the master batch (referenced as Part A below). The master batch pellets were admixed with ethylene methacrylic acid (DuPont Nucrel®) (referenced as Part B below) in a 1:1 ratio and three to five mil monolayer films were blown using a Killion Lab Line having a 2.5 cm blown die with a single lip air ring and a die gap setting of 0.064 cm. The blow up ratio was varied from 1.2:1 to 4:1 and corona treatment was not used. The films preparation conditions are indicated in Table 21. The films were prepared in both tube and single wound sheet form and were stored in sealed bags.

The films were analyzed for NaClO₂, NaClO₃ and NaCl content with the results reported in Table 21 in weight percent. The films were analyzed for ClO₂ release with the average results for two runs reported in Table 22.

TABLE 21

| | Melt Extrusion | | | | |
|---|---|---|---|---|---|
| | Temperature | Film Melt | | | |
| | for Part A | Temperature for | NaClO₂ | NaCl | NaClO₃ |
| Film | (° C.) | Parts A & B (° C.) | (wt. %) | (wt. %) | (wt. %) |
| 1 | 96 | 93 | 0.85 | 0.78 | 0.19 |
| 2 | 107 | 132 | 0.84 | 1.02 | 0.22 |
| 3 | 107 | 121 | 1.37 | 0.67 | 0.13 |
| 4 | 107 | 93 | 1.33 | 0.95 | 0.18 |
| 5 | 107 | 132 | 1.26 | 0.64 | 0.12 |
| 6 | 118 | 93 | 0.96 | 0.83 | 0.17 |
| 7 | 118 | 121 | 0.73 | 0.91 | 0.21 |
| 8 | 118 | 132 | 0.66 | 1.05 | 0.26 |

TABLE 22

| | ppm | | | | |
|---|---|---|---|---|---|
| | ClO₂ | ppm ClO2/g | Peak Time | ppm ClO2 | ppm ClO2/g film |
| Film | (peak) | film | (h) | (at 5 days) | (at 5 days) |
| 1 | 0.1 | 1 | 21 | 0 | 0 |
| 2 | 2.3 | 23 | 17 | 0.6 | 6 |
| 3 | 2.6 | 22.5 | 21 | 0.75 | 6.5 |
| 4 | 0.35 | 9.5 | 5 | 0 | 0 |
| 5 | 0.1 | 4.5 | 21 | 0.05 | 0.5 |
| 6 | 0.3 | 8 | 16 | 0.1 | 0.4 |
| 7 | 0.1 | 1 | 16 | 0 | 0 |
| 8 | 1.2 | 12 | 17 | 0.4 | 4 |

While the invention is susceptible to various modifications and alternative forms, specific embodiments thereof have been shown by way of example and have been described herein in detail. It should be understood, however, that it is not intended to limit the invention to the particular form disclosed, but on the contrary, the intention is to cover all modifications, equivalents and alternatives falling within the spirit and scope of the invention as defined in the appended claims.

In view of the above, it will be seen that the several objects of the invention are achieved and other advantageous results attained.

As various changes could be made in the above methods without departing from the scope of the invention, it is intended that all matter contained in the above description or shown in the accompanying drawings shall be interpreted as illustrative and not in a limiting sense.

When introducing elements of the present invention or the preferred embodiments thereof, the articles “a”, “an”, “the”, and “said” are intended to mean that there are one or more of the elements. The terms “comprising”, “including,” and “having” are intended to be inclusive and mean that there may be additional elements other than the listed elements.

1. A compatible polymer blend for retarding bacterial, fungal and viral contamination and mold growth comprising:
anions capable of reacting with hydronium ions to generate a gas;
a hydrophilic polymer having a glass transition temperature of less than 100° C.; and
either a hydrophobic polymer and an acid releasing agent, or an acid releasing hydrophobic polymer,
the compatible polymer blend being substantially free of water and capable of generating and releasing the gas upon hydration of the acid releasing agent or the acid releasing hydrophobic polymer.

2. The compatible polymer blend of claim 1 wherein the hydrophobic and hydrophilic polymers form an interpenetrating network upon hydration.

3. The compatible polymer blend of claim 1 wherein the compatible polymer blend is capable of being melt processed at a temperature from about 90° C. to about 150° C. by extrusion molding, compression molding, blow molding, or injection molding.

4. The compatible polymer blend of claim 1 wherein the hydrophobic polymer is a copolymer or terpolymer formed from methylene, ethylene, polypropylene, imides, vinyl chloride or vinyl alcohol and at least one acid releasing monomer.

5. The compatible polymer blend of claim 1 wherein the hydrophobic polymer is polyoxazoline having the formula: 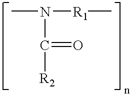
wherein R₁ is a substituted or unsubstituted alkylene group containing from 1 to 4 carbon atoms; R₂ is a substituted or unsubstituted aryl group or a substituted or unsubstituted alkyl group containing from 1 to 6 carbon atoms; and wherein n is an integer which provides the polymer with a molecular weight of less than about 100,000 daltons.

6. The compatible polymer blend of claim 1 wherein the anions are selected from the group consisting of chlorite, chloride, bisulfite, sulfite, bicarbonate, nitrite, cyanide, sulfide, hydrosulfide and hypochlorite.

7. A compatible polymer blend for retarding bacterial, fungal and viral contamination and mold growth comprising:
anions capable of reacting with hydronium ions to generate a gas;
a hydrophilic polymer having the structure: 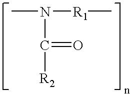
wherein R₁ is a substituted or unsubstituted alkylene group containing from 1 to 4 carbon atoms; R₂ is a substituted or unsubstituted aryl group or a substituted or unsubstituted alkyl group containing from 1 to 6 carbon atoms; and wherein n is an integer which provides the polymer with a molecular weight of less than about 100,000 daltons; and
either a hydrophobic polymer and an acid releasing agent, or an acid releasing hydrophobic polymer,
the compatible polymer blend being substantially free of water and capable of generating and releasing the gas upon hydration of the acid releasing agent or the acid releasing hydrophobic polymer.

8. The compatible polymer blend of claim 7 wherein the hydrophilic and hydrophobic polymers form an interpenetrating network upon hydration.

9. The compatible polymer blend of claim 7 wherein the compatible polymer blend is processed at a temperature from about 90° C. to about 150° C. by extrusion molding, compression molding, blow molding or injection molding.

10. The compatible polymer blend of claim 7 wherein the acid releasing hydrophobic polymer is a copolymer or terpolymer comprising methylene, ethylene, polypropylene, imide, vinyl chloride or vinyl alcohol and at least one acid releasing monomer.

11. The compatible polymer blend of claim 7 wherein the anions are selected from the group consisting of chlorite, chloride, bisulfite, sulfite and bicarbonate.

12. The compatible polymer blend of claim 1 wherein the compatible polymer blend is capable of phase separation to form an interpenetrating network and generating and releasing the gas upon hydration of the acid releasing hydrophobic polymer.

13. The compatible polymer blend of claim 1 further comprising
an upper moisture regulating layer in contact with an upper surface of the compatible polymer blend, and a lower moisture regulating layer in contact with a lower surface of the compatible polymer blend thereby forming a multilayered composite, wherein moisture permeating the upper or lower moisture regulating layers hydrates the compatible polymer blend to generate and release a gas from the multilayered composite.

14. The compatible polymer blend of claim 13 wherein the upper or lower moisture regulating layer comprises a polyvinylchloride polymer.

15. A process for preparing a compatible polymer blend having a melt temperature less than about 150° C., the process comprising:
forming a mixture or slurry of a liquid, anions capable of reacting with hydronium ions to generate a gas, and a hydrophilic polyoxazoline polymer of the general formula: 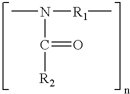
wherein R₁ is a substituted or unsubstituted alkylene group containing from 1 to 4 carbon atoms; R₂ is a substituted or unsubstituted aryl group or a substituted or unsubstituted alkyl group containing from 1 to 6 carbon atoms; and wherein n is an integer which provides the polymer with a molecular weight of less than about 100,000 daltons;
removing the liquid to form a glass; and
melt blending the glass with either a hydrophobic polymer and an acid releasing agent, or an acid releasing hydrophobic polymer to form the compatible polymer blend.

16. The process of claim 15 wherein the liquid comprises water or methanol.

17. The process of claim 15 wherein the anions comprise chlorite.

18. The process of claim 15 wherein the mixture further comprises an alkali hydroxide.

19. The process of claim 15 wherein the polyoxazoline polymer comprises polyethyl oxazoline; and the acid releasing agent comprises an alkali hydrogen phosphate, an alkali hydrogen polyphosphate, a phosphosilicic anhydride, a phosphosilicic anhydride fatty acid ester or an alkenyl succinic anhydride, or the acid releasing hydrophobic polymer is a copolymer or terpolymer comprising methylene, ethylene, polypropylene, imide, vinyl chloride or vinyl alcohol and at least one acid releasing monomer.

20. The process of claim 15 wherein the compatible polymer blend further comprises an olefin or paraffin wax and the blend is melt processed to form an object or film.

21. A process for preparing a compatible polymer blend having a melt temperature less than about 150° C., the process comprising:
providing a mixture comprising anions capable of reacting with hydronium ions to generate a gas, a hydrophilic polyoxazoline polymer and either a hydrophobic polymer and an acid releasing agent, or an acid releasing hydrophobic polymer;
melt processing the mixture to form the compatible polymer blend,
wherein the hydrophilic polyoxazoline polymer has the formula: 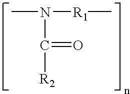
wherein R₁ is a substituted or unsubstituted alkylene group containing from 1 to 4 carbon atoms; R₂ is a substituted or unsubstituted aryl group or a substituted or unsubstituted alkyl group containing from 1 to 6 carbon atoms; and wherein n is an integer which provides the polymer with a molecular weight of less than about 100,000 daltons.

22. The process of claim 21 wherein the anions comprise chlorite.

23. The process of claim 21 wherein the polyoxazoline polymer is polyethyl oxazoline; and the acid releasing agent comprises an alkali hydrogen phosphate, an alkali hydrogen polyphosphate, a phosphosilicic anhydride, a phosphosilicic anhydride fatty acid ester or an alkenyl succinic anhydride, or the acid releasing hydrophobic polymer is a copolymer or terpolymer comprising methylene, ethylene, polypropylene, imide, vinyl chloride or vinyl alcohol and at least one acid releasing monomer.

24. The process of claim 21 wherein the compatible polymer blend further comprises an olefin or paraffin wax and the blend is melt processed to form an object or film.

25. A method of retarding bacterial, fungal, and viral contamination and growth of molds on a surface and/or deodorizing the surface comprising:
melt processing the compatible polymer blend of claim 1 to form an object or film; and
exposing the surface of the object or film to moisture to release the gas from the compatible polymer blend into the atmosphere surrounding the surface to retard bacterial, fungal, and viral contamination and growth of molds on the surface and/or deodorize the surface.

26. The method of claim 25 wherein the compatible polymer blend is optically transparent.

27. The method of claim 25 wherein the object or film is melt processed at a temperature from about 90° C. to about 150° C. by extrusion molding, compression molding, blow molding or injection molding.
